(19) United States
(12) Patent Application Publication
(10) Pub. No.: US 2007/0166227 A1
(43) Pub. Date: Jul. 19, 2007

(54) Crowned dithiocarbamate metal complexes and methods for their use

(76) Inventors: Shuang Liu, West Lafeyette, IN (US); Zhengjie He, West Lafayette, IN (US)

Correspondence Address: BRINKS HOFER GILSON & LIONE, P.O. BOX 10395, CHICAGO, IL, 60610, US

(21) Appl. No.: 10/588,343

(22) PCT Filed: Feb. 10, 2005
(86) PCT No.: PCT/US05/04872

§ 371 Date: Aug. 2, 2006

Related U.S. Application Data
(60) Provisional application No. 60/543,176, filed on Feb. 10, 2004.

(51) Int. Cl. A61K 51/00; C07F 13/00

(52) U.S. Cl. 424/1.11; 534/14

(57) ABSTRACT

Compositions containing crowned dithiocarbamate metal complexes and methods of using these compositions; neutral and cationic radioactive metal-nitrido complexes of crowned dithiocarbamates (DTCs) and methods of using these complexes as radiopharmaceuticals for diagnosis and treatment of cardiovascular disorders, infectious diseases, and cancer; tripodal chelatormetal complexes of crowned DTCs and methods of using these complexes for treating diseases such as those characterized by nitric oxide overproduction; and methods of using crowned DTCs for heavy metal detoxification are described.

### RELATED APPLICATIONS

This application claims the benefit of U.S. Provisional Application No. 60/543,176, filed Feb. 10, 2004, the entire contents of which are incorporated herein by reference, except that in the event of any inconsistent disclosure or definition from the present application, the disclosure or definition herein shall be deemed to prevail.

### FIELD OF THE INVENTION

The present invention relates to compositions containing crowned dithiocarbamate metal complexes and to methods for their use.

### BACKGROUND

Since the early 1980s, extensive research efforts have been directed towards the development of lipophilic ^{99m}Tc complex cations as heart imaging agents (Nun, A. D. Semin. Nucl. Med. 1990, 20, 111). As a result of these efforts, two cationic ^{99m}Tc complexes (^{99m}Tc-Sestamibi and ^{99m}Tc-Tetrofosmin) have been approved as commercial radiopharmaceuticals for myocardial perfusion imaging. Q3 and Q12 are cationic ^{99m}Tc complexes containing two monodentate phosphine ligands and a tetradentate Schiff-base chelator. Lipophilic ^{99m}Tc complexes, such as ^{99m}Tc—N-Noet, with neutral charges have also been studied for myocardial perfusion imaging. ^{99m}Tc—N-Noet is still under clinical investigation in Europe.

Perfusion refers to blood flow at the cellular level, such as the delivery of nutrients and removal of waste products to maintain cellular function (Dilsizian, V. J. Nucl. Cardiol. 2000, 7, 180; Marmion M. and Deutsch, E. Quart. J. Nucl. Med. 2000, 7, 701). An ideal myocardial perfusion agent has a high first-pass extraction with stable myocardial retention, which linearly tracks myocardial blood flow over a wide range. Hepatic and gastrointestinal uptake should be minimal with exercise as well as with pharmacological stress and rest studies. The agent may redistribute; but should do so in a predictable and reliable manner (Saha, G. B. et al. Nucl. Med. Biol. 1992, 19, 1; Jain, D. Semin. Nucl. Med. 1999, 29, 221; Banerjee, S. et al. Semin. Nucl. Med. 2001, 31, 260). Despite the widespread use of ^{99m}Tc-Sestamibi and ^{99m}Tc-Tetrofosmin in myocardial perfusion imaging studies, they do not meet the requirements of an ideal perfusion imaging agent mainly due to the low first-pass extraction, flow-dependence and high uptake in liver and lungs. Therefore, there is still a continuing need for the development of better radiotracers for myocardial perfusion imaging.

### SUMMARY

The scope of the present invention is defined solely by the appended claims, and is not affected to any degree by the statements within this summary.

By way of introduction, a first composition embodying features of the present invention includes a compound having a formula (M≡N)L¹ and pharmaceutically acceptable salts thereof, wherein N is nitrogen, M is a transition metal, and L¹ is a first crowned dithiocarbamate. The first crowned dithiocarbamate includes a first crown ether-containing group of formula [(CH₂)ₐ—O]_{b}—(CH₂)_{c}, wherein a is at least 2, b is at least 3, and c is at least 2.

A second composition embodying features of the present invention includes a compound having a formula

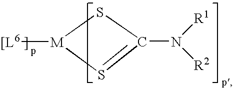

and pharmaceutically acceptable salt thereof, wherein: M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II); p and p′ are integers and are independently selected from 0-2; R¹ and R² contain a crown ether-containing group of formula [(CH₂)ₐ—O]_{b}—(CH₂)_{c}, wherein a is at least 2, b is at least 3, and c is at least 2, or wherein R¹ and R² together contain the crown ether-containing group; and L⁶ is a tripodal chelator with a formula selected from the group consisting of:

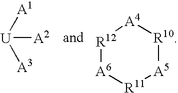

U is selected from the group consisting of R¹³B, CR¹³, and P(═O). A¹, A² and A³ are imine-N containing heterocycles. A⁴, A⁵ and A⁶ are selected from the group consisting of NR¹⁰, PR¹⁰, and S. R¹⁰, R¹¹ and R¹² are selected from a group of formula —(CH₂)_{g}—, wherein g is 2-5. R¹³ is selected from the group consisting of H, alkyl and aryl. R¹⁴ is selected from the group consisting of H, alkyl, aryl, and alkoxyalkyl.

A method for radioimaging a subject embodying features of the present invention includes: (a) providing (i) a subject and (ii) a composition containing a compound having a formula (M≡N)L¹ and pharmaceutically acceptable salts thereof; (b) administering the composition to the subject; and c) scanning at least a portion of the subject using a radioimaging device. In the formula, N is nitrogen, M is a radioactive transition metal, and L¹ is a first crowned dithiocarbamate. The first crowned dithiocarbamate contains a first crown ether-containing group of formula [(CH₂)ₐ—O]_{b}—(CH₂)_{c}, wherein a is at least 2, b is at least 3, and c is at least 2.

A method of treating a disease resulting from overproduction of nitric oxide or reactive oxygen species embodying features of the present invention includes: (a) providing (i) a subject with a disease and (ii) a composition containing a compound having a formula:

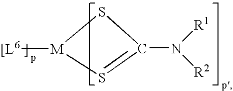

and pharmaceutically acceptable salt thereof; and b) administering the composition to the subject. M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II). The subscripts p and p′ are integers and are independently selected from 0-2. R¹ and R² contains a crown ether-containing group of formula [(CH₂)ₐ—O]_{b}—(CH₂)_{c}, wherein a is at least 2, b is at least 3, and c is at least 2, or wherein R¹ and R² together contain the crown ether-containing group. L⁶ is a tripodal chelator with a formula selected from the group consisting of:

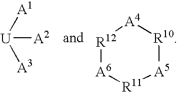

U is selected from the group consisting of R¹³B, CR¹³, and P(═O). A¹, A² and A³ are imine-N containing heterocycles. A⁴, A⁵ and A⁶ are selected from the group consisting of NR¹⁰, PR¹⁰, and S. R¹⁰, R¹¹ and R¹² are selected from a group of formula —(CH₂)_{g}—, wherein g is 2-5. R¹³ is selected from the group consisting of H, alkyl and aryl. R¹⁴ is selected from the group consisting of H, alkyl, aryl, and alkoxyalkyl.

A method of treating metal poisoning embodying features of the present invention includes (a) providing (i) a subject with metal poisoning, and (ii) a composition containing a crowned dithiocarbamate; and (b) administering the composition to the subject. The crowned dithiocarbamate includes a crown ether-containing group of formula [(CH₂)ₐ—O]_{b}—(CH₂)_{c}, wherein a is at least 2, b is at least 3, and c is at least 2.

### DETAILED DESCRIPTION

Compositions embodying features of the present invention contain crowned dithiocarbamate metal complexes. Representative compositions and methods for their use are described hereinbelow. Compositions embodying features of the present invention provide neutral and cationic radioactive metal-nitrido complexes of crowned dithiocarbamates (DTCs). Methods embodying features of the present invention include using these complexes as radiopharmaceuticals for diagnosis and treatment of cardiovascular disorders, infectious disease, and cancer. The present invention also provides tripodal chelator-metal complexes of crowned DTCs and methods of using these complexes for treating diseases such as those characterized by nitric oxide overproduction. The present invention further provides methods of using crowned DTCs for heavy metal detoxification.

The detailed description hereinbelow provides further details on the compositions, kits, and methods described above, and is organized in the following sections: DEFINITIONS, EXEMPLARY PHARMACEUTICAL COMPOSITIONS; THERAPEUTIC USES; METHODS OF MAKING CROWNED DITHIOCARBAMATE METAL COMPLEXES; and KITS, THERAPEUTICS COMPOSITIONS AND ROUTES OF ADMINISTRATION. All literature publications and patent documents (both domestic and foreign) cited in this specification are hereby incorporated by reference herein in their entireties, except that in the event of any inconsistent disclosure or definition from the present application, the disclosure or definition herein shall be deemed to prevail.

Definitions

Throughout this description and in the appended claims, the following definitions are to be understood:

The terms “subject” and “patient” refer to any animal, such as a mammal. Representative examples include but are not limited to humans, dogs, cats, birds, livestock, and the like.

The term “substituted” refers to the replacement of any one or more hydrogens on the designated atom with a selection from the indicated group, provided that the designated atom's normal valency is not exceeded and that the substitution results in a stable compound. When a substituent is keto (i.e., ═O), then two hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part of (i.e., within) the ring.

Compounds in accordance with the present invention are intended to include all isotopes of atoms. Isotopes include those atoms having the same atomic number but different mass numbers. By way of general example and without limitation, isotopes of hydrogen include tritium and deuterium. Isotopes of carbon include C-13 and C-14. When any variable (e.g., R⁶) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are preferably stable compounds. When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent.

The term “alkyl” refers to both branched and straight-chain saturated aliphatic hydrocarbon groups having the specified number of carbon atoms. Representative examples of alkyl include but are not limited to methyl, ethyl, n-propyl, i-propyl, n-butyl, s-butyl, t-butyl, n-pentyl, s-pentyl, and the like.

The term “haloalkyl” refers to both branched and straight-chain saturated aliphatic hydrocarbon groups having the specified number of carbon atoms, substituted with 1 or more halogen atoms (e.g., —CᵥF_{w} where v=1 to 3 and w=1 to (2v+1)). Representative examples of haloalkyls include but are not limited to trifluoromethyl, trichloromethyl, pentafluoroethyl, pentachloroethyl, and the like.

The term “alkoxy” refers to an alkyl group as defined above with the indicated number of carbon atoms attached through an oxygen bridge. Representative examples of alkoxys include but are not limited to methoxy, ethoxy, n-propoxy, i-propoxy, n-butoxy, s-butoxy, t-butoxy, n-pentoxy, s-pentoxy, and the like.

The term “cycloalkyl” refers to saturated ring groups, including but not limited to cyclopropyl, cyclobutyl, cyclopentyl, cyclohexyl, and the like.

The term “alkenyl” refers to hydrocarbon chains of either a straight or branched configuration containing one or more unsaturated carbon-carbon bonds which may occur in any stable point along the chain. Representative examples include but are not limited to ethenyl, propenyl, and the like.

The term “alkynyl” refers to hydrocarbon chains of either a straight or branched configuration containing one or more triple carbon-carbon bonds which may occur in any stable point along the chain. Representative examples include but are not limited to ethynyl, propynyl, and the like.

The terms “heterocycle” and “heterocyclic system” refer to a stable 5- to 7-membered monocyclic or bicyclic ring or a 7- to 10-membered bicyclic heterocyclic ring which is saturated, partially unsaturated or unsaturated (e.g., aromatic), and which contains carbon atoms and from 1 to 4 heteroatoms independently selected from the group consisting of N, O and S. These terms include any bicyclic group in which any of the above-defined heterocyclic rings is fused to a benzene ring. The nitrogen and sulfur heteroatoms may optionally be partially or completely oxidized. The heterocyclic ring may be attached to its pendant group at any heteroatom or carbon atom (preferably resulting in a stable structure). The heterocyclic rings described herein may be substituted on carbon or on a nitrogen atom. A nitrogen in the heterocycle may optionally be quaternized. It is preferred that when the total number of S and O atoms in the heterocycle exceeds 1 that the heteroatoms are not adjacent to one another. In some embodiments, it is preferred that the total number of S and O atoms in the heterocycle is not more than 1.

The terms “aromatic heterocyclic system” and “heteroaryl” refer to a stable 5- to 7-membered monocyclic or bicyclic ring or a 7- to 10-membered bicyclic heterocyclic aromatic ring which contains carbon atoms and from 1 to 4 heteroatoms independently selected from the group consisting of N, O and S. In some embodiments, it is preferred that the total number of S and O atoms in the aromatic heterocycle is not more than 1.

Representative heteroatom-containing rings for use in accordance with the present invention include but are not limited to: acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, and the like. In some embodiments, preferred heterocycles include but are not limited to: pyridinyl, furanyl, thienyl, pyrrolyl, pyrazolyl, pyrrolidinyl, imidazolyl, indolyl, benzimidazolyl, 1H-indazolyl, oxazolidinyl, benzotriazolyl, benzisoxazolyl, oxindolyl, benzoxazolinyl, isatinoyl, and the like. Fused ring and spiro compounds containing, for example, one or more of the above-described representative heterocycles are also contemplated for use in accordance with the present invention.

The phrase “pharmaceutically acceptable” refers to those compounds, materials, compositions, and/or dosage forms which are, within the scope of sound medical judgment, suitable for use with the tissues of human beings and animals without resulting in excessive toxicity, irritation, allergic response, or other problems or complications, commensurate with a reasonable benefit/risk ratio.

The phrase “pharmaceutically acceptable salts” refers to derivatives of the disclosed compounds wherein the parent compound is modified by making acid or base salts thereof. Representative examples of pharmaceutically acceptable salts include but are not limited to: mineral or organic acid salts of basic residues, such as amines; and alkali or organic salts of acidic residues, such as carboxylic acids. Pharmaceutically acceptable salts in accordance with the present invention include conventional non-toxic salts or the quaternary ammonium salts of the parent compound formed, for example, from non-toxic inorganic or organic acids. For example, such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acids.

Pharmaceutically acceptable salts in accordance with the present invention may be synthesized from a parent compound containing a basic or acidic moiety by conventional chemical methods. Typically, such salts may be prepared by reacting the free acid or base forms of these compounds with a substantially stoichiometric amount and/or a slight excess of the appropriate base or acid in water or in an organic solvent, or in a mixture of the two. In some embodiments, nonaqueous media such as ether, ethyl acetate, ethanol, isopropanol, and acetonitrile are preferred. Lists of suitable salts are found in Remington's Pharmaceutical Sciences, 17th ed., Mack Publishing Company, Easton, Pa., 1985, p. 1418.

Since prodrugs are known to enhance numerous desirable qualities of pharmaceuticals (e.g., solubility, bioavailability, manufacturing, etc.) the compounds of the present invention may be delivered in prodrug forms. Thus, the present invention includes prodrugs of compounds embodying features of the present invention, methods of delivering the same, and compositions containing the same. The term “prodrugs” refers to any covalently bonded carriers which release an active parent drug of the present invention in vivo when such prodrug is administered to a mammalian subject. Prodrugs in accordance with the present invention may be prepared by modifying functional groups present in the compound in such a way that the modifications are cleaved, either in routine manipulation or in vivo, to the parent compound. Representative prodrugs include compounds embodying features of the present invention, wherein a hydroxy, amino, or sulfhydryl group is bonded to any group such that when the prodrug is administered to a mammalian subject, it cleaves to form a free hydroxyl, free amino, or free sulfhydryl group, respectively. Representative examples of prodrugs include but are not limited to acetate, formate and benzoate derivatives of alcohol and amine functional groups in compounds embodying features of the present invention.

The phrases “stable compound” and “stable structure” refer to compounds and structures that are sufficiently robust to survive isolation to a useful degree of purity from a reaction mixture, and formulation into an efficacious therapeutic agent. The coordination sphere of the radionuclide includes all the ligands or groups bound to the radionuclide. For a transition metal radionuclide to be stable, it typically has a coordination number (number of donor atoms) comprised of an integer greater than or equal to 4 and less than or equal to 7 (i.e., there are 4 to 7 atoms bound to the metal and it is said to have a complete coordination sphere). The requisite coordination number for a stable radionuclide complex is determined by the identity of the radionuclide, its oxidation state, and the type of donor atoms.

Exemplary Pharmaceutical Compositions

In some embodiments, the present invention provides compositions comprising a compound comprising a formula (M≡N)L¹ and pharmaceutically acceptable salts thereof, wherein N is nitrogen, M is a transition metal, and L¹ is a first crowned dithiocarbamate. The first crowned dithiocarbamate comprises a first crown ether-containing group of formula [(CH₂)ₐ—O]_{b}—(CH₂)_{c}, wherein a is at least 2, b is at least 3, and c is at least 2. In some embodiments, the transition metal is covalently bound to the first crowned dithiocarbamate.

In some embodiments, the first crowned dithiocarbamate comprises a formula:

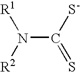

and pharmaceutically acceptable salt thereof, wherein R¹ or R² comprises the first crown ether-containing group, or R¹ and R² together comprise the first crown ether-containing group.

In some embodiments, the compound further comprises L² and comprises a formula (M≡N)L¹L² and pharmaceutically acceptable salts thereof, wherein L² is a second crowned dithiocarbamate. The second crowned dithiocarbamate comprises a second crown ether-containing group of formula [(CH₂)ₐ—O]_{b}—(CH₂)_{c}, wherein a is at least 2, b is at least 3, and c is at least 2. In some embodiments, the transition metal is covalently bound to the first and second crowned dithiocarbamates.

In some embodiments, the second crowned dithiocarbamate comprises a formula:

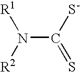

and pharmaceutically acceptable salt thereof, wherein R¹ or R² comprises the second crown ether-containing group, or R¹ and R² together comprise the second crown ether-containing group.

In some embodiments, the compound further comprises L³, L⁴, and L⁵ and comprises a formula: 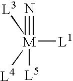
and pharmaceutically acceptable salts thereof, wherein L³, L⁴, and L⁵ each comprises an isonitrile of formula:

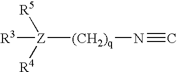

wherein q is 0-3, and Z is carbon or silicon. R³, R⁴ and R⁵ are the same or different, and are selected from the group consisting of H, C₁-C₁₀ alkyl substituted with 0-5 R⁶, aryl substituted with 0-5 R⁶, heteroaryl substituted with 0-5 R⁶, and macrocyclic crown ether containing 2-8 ether-oxygen atoms. R⁶ is selected from the group consisting of H, OH, OR⁷, C(═O)OR⁷, C(═O)NR⁸R⁹, PO(OR⁸)₂, PO(NR⁸R⁹)₂ and SO₂R⁷; R⁷, R⁸, and R⁹ are the same or different, and are selected from the group consisting of H, alkyl, aryl, and heteroaryl, or R⁸ and R⁹ together form a macrocyclic crown ether containing 2-8 ether-oxygen atoms.

In some embodiments, the compound further comprises L³, L⁴, and L⁵ and comprises a formula:

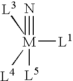

and pharmaceutically acceptable salts thereof, wherein L³, L⁴, and L⁵ together form a tripodal chelator of formula:

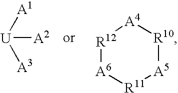

wherein U is selected from the group consisting of R¹³B, CR¹³, and P(═O); A¹, A² and A³ are imine-N containing heterocycles; A⁴, A⁵ and A⁶ are selected from the group consisting of NR¹⁴, PR¹⁴, S, and O; R¹⁰, R¹¹ and R¹² are selected from a group of formula:
—(CH₂)_{g}—,

wherein g is 2-5; R¹³ is selected from the group consisting of H, alkyl and aryl group; and R¹⁴ is selected from the group consisting of H, alkyl, aryl, and alkoxyalkyl.

In some embodiments, the transition metal is a radioactive metal. In some embodiments, the transition metal is ^{99m}Tc or ^{94m}Tc. In other embodiments, the transition metal is ¹⁸⁶Re or ¹⁸⁸Re. In some embodiments, subscript a in the formula of the first and/or second crown ether-containing group is 2, 3, 4, or 5. In other embodiments, subscript a in the formula of the first and/or second crown ether-containing group is 2 or 3. In some embodiments, subscript a in the formula of the first and/or crown ether-containing group is 2. In other embodiments, subscript b in the formula of the first and/or second crown ether-containing group is 3, 4, 5, 6, 7, or 8. In additional embodiments, subscript b in the formula of the first and/or second crown ether-containing group is 3, 4, 5 or 6. In further embodiments, subscript c in the formula of the first and/or second crown ether-containing group is 2, 3, 4, or 5. In some embodiments, subscript c in the formula of the first and/or second crown ether-containing group is 2 or 3. In some embodiments, subscript c in the formula of the first and/or second crown ether-containing group is 2.

In some compounds embodying features of the present invention, M is ^{99m}Tc; a is 2 or 3; b is 3-6; c is 2 or 3; q is 1; Z is carbon; R³, R⁴ and R⁵ are the same or different, and are selected from the group consisting of H, C₁-C₅ alkyl substituted with a R⁶, aryl substituted with a R⁶, heteroaryl substituted with a R⁶, and macrocyclic crown ether containing 3-6 ether-oxygen atoms. R⁶ is selected from the group consisting of OR⁸, C(═O)OR⁸, C(═O)NR⁸R⁹, and PO(NR⁸R⁹)₂. R⁷, R⁸ and R⁹ are the same or different, and are selected from the group consisting of H, alkyl, aryl, and heteroaryl, or R⁸ and R⁹ may be taken together to form a macrocyclic crown ether containing 3-6 ether-oxygen atoms. A¹, A² and A³ are selected from the group consisting of imidazolyl, pyrazolyl, oxazolinyl, methimazolyl, and pyridyl. A⁴, A⁵ and A⁶ are selected from the group consisting of NR¹⁰, PR¹⁰, and S. The subscript g is 2 or 3. R¹³ is selected from the group consisting of H, methyl, and phenyl. R¹⁴ is selected from the group consisting of ethyloxyethyl, ethoxylpropyl, methyoxyethyl, and methoxypropyl.

In some embodiments, the first and/or second crowned dithiocarbamate comprises

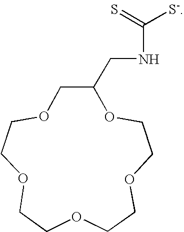

In some embodiments, the first and/or second crowned dithiocarbamate comprises

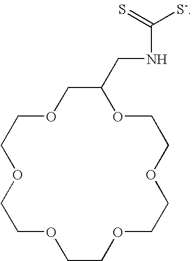

In some embodiments, the first and/or second crowned dithiocarbamate comprises

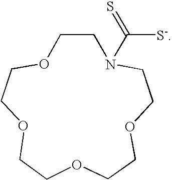

In some embodiments, the first and/or second crowned dithiocarbamate comprises

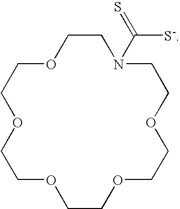

In some embodiments, the first and/or second crowned dithiocarbamate comprises

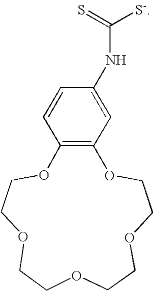

In some embodiments, the first and/or second crowned dithiocarbamate comprises

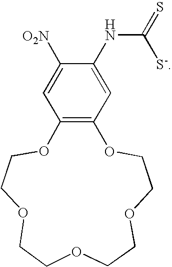

In some embodiments, the first and/or second crowned dithiocarbamate is selected from the group consisting of

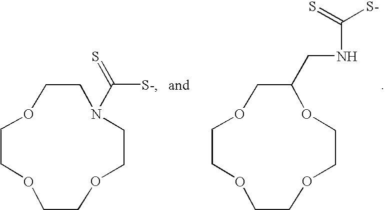

In some embodiments, the compound is selected from the group consisting of:

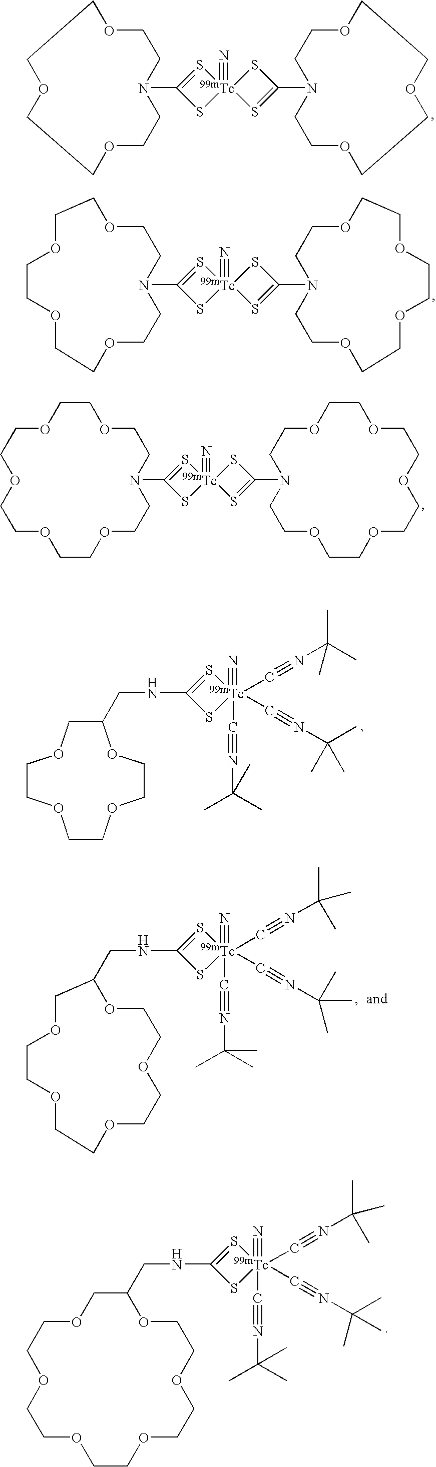

In some embodiments, the compound is selected from the group consisting of:

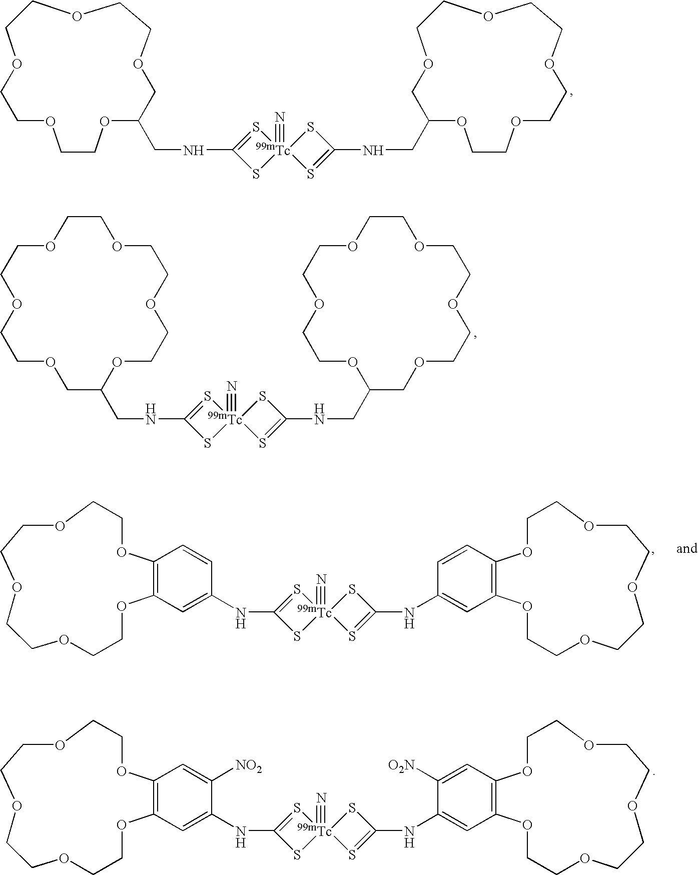

In embodiments including a tripodal chelator, the tripodal chelator is selected from the group consisting of

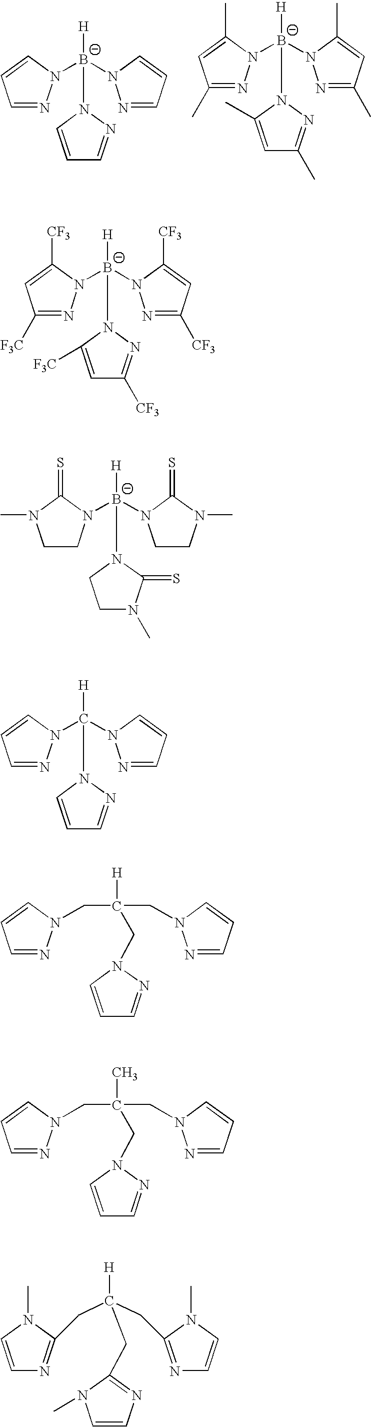

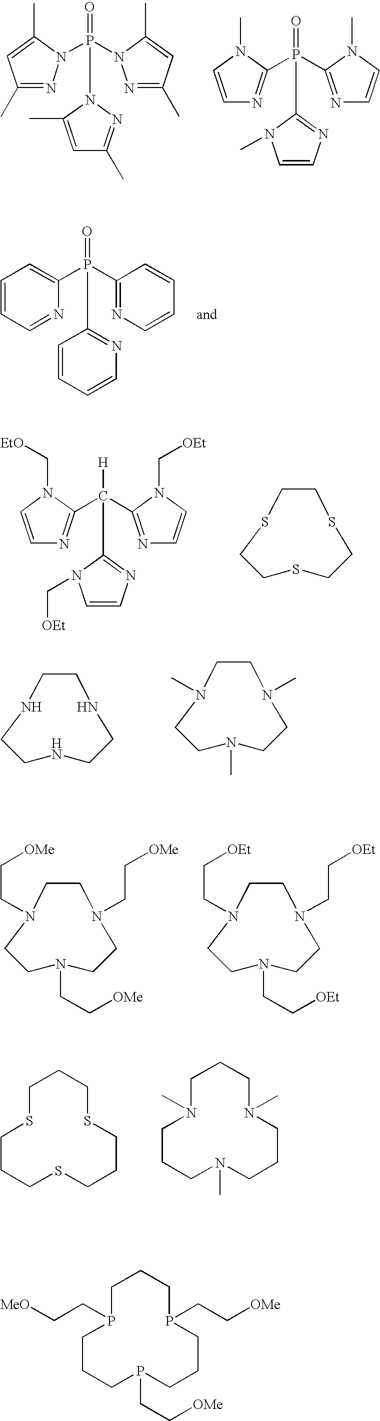

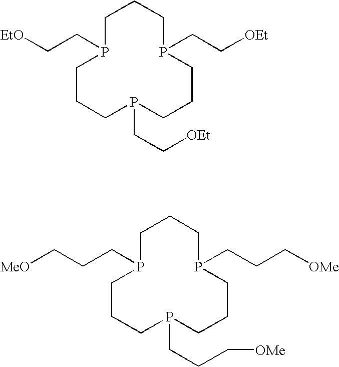

In some embodiments, the present invention provides compositions comprising a formula:

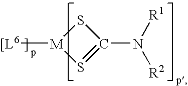

and pharmaceutically acceptable salt thereof, wherein M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II); p and p′ are integers and are independently selected from the group consisting of 0-2. R¹ and R² comprise a crown ether-containing group of formula [(CH₂)ₐ—O]_{b}—(CH₂)_{c}, wherein a is at least 2, b is at least 3, and c is at least 2, or wherein R¹ and R² together comprise the crown ether-containing group. L⁶ is a tripodal chelator with a formula selected from the group consisting of:

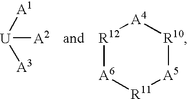

wherein U is selected from the group consisting of R¹³B, CR¹³, and P(═O). A¹, A² and A³ are imine-N containing heterocycles. A⁴, A⁵ and A⁶ are selected from the group consisting of NR¹⁰, PR¹⁰, and S. R¹⁰, R¹¹ and R¹² are selected from a group of formula:
—(CH₂)_{g}—,

wherein g is 2-5. R¹³ is selected from the group consisting of H, alkyl and aryl group. R¹⁴ is selected from the group consisting of H, alkyl, aryl, and alkoxyalkyl.

In some embodiments, M is Ru(III), p is 1, p′ is 2, R¹ and R² are the same or different and are selected from a macrocyclic crown ether-containing group, or R¹ and R² are taken together to form a macrocycle of formula [(CH₂)ₐ—O]_{b}—(CH₂)_{c}, wherein a is 2 or 3, b is 3-6, and c is 2 or 3. A¹, A² and A³ are selected from the group consisting of imidazolyl, pyrazolyl, oxazolinyl, methimazolyl, and pyridyl. A⁴, A⁵ and A⁶ are selected from the group consisting of NR¹⁰, PR¹⁰, and S. The subscript g is 2 or 3. R¹³ is selected from the group consisting of H, methyl, and phenyl. R¹⁴ is selected from the group consisting of ethyloxyethyl, ethoxylpropyl, methyoxyethyl, and methoxypropyl. R¹⁴ is selected from the group consisting of H, alkyl, aryl, and alkoxyalkyl.

In some embodiments, the present invention provides methods for radioimaging a subject (e.g., a human) comprising: a) providing a subject, and a composition comprising a compound of formula (M≡N)L¹ and pharmaceutically acceptable salts thereof; b) administering the composition to the subject, and c) scanning at least a portion of the subject using a radioimaging device. In the formula, N is nitrogen, M is a radioactive transition metal, and L¹ is a first crowned dithiocarbamate, wherein the first crowned dithiocarbamate comprises a first crown ether-containing group of formula [(CH₂)ₐ—O]_{b}—(CH₂)_{c}, wherein a is at least 2, b is at least 3, and c is at least 2; In some embodiments, at least a portion of the subject is tissue suspected of being diseased. In other embodiments, the at least a portion of the subject is myocardial tissue. In some embodiments, the subject is a mammal (e.g., a human, cat, dog, pig, horse or the like).

In some embodiments, the present invention provides methods of treating a disease resulting from overproduction of nitric oxide or reactive oxygen species, comprising; a) providing a subject with a disease, and a composition comprising a compound of formula:

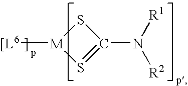

and pharmaceutically acceptable salt thereof; and b) administering the composition to the subject. In the formula, M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II). The subscripts p and p′ are integers and are independently selected from 0-2. R¹ and R² comprise a crown ether-containing group of formula [(CH₂)ₐ—O]_{b}—(CH₂)_{c}, wherein a is at least 2, b is at least 3, and c is at least 2, or wherein R¹ and R² together comprise the crown ether-containing group. L⁶ is a tripodal chelator with a formula selected from:

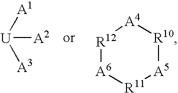

wherein U is selected from the group consisting of R¹³B, CR¹³, and P(═O). A¹, A² and A³ are imine-N containing heterocycles. A⁴, A⁵ and A⁶ are selected from the group consisting of NR¹⁰, PR¹⁰, and S. R¹⁰, R¹¹ and R¹² are selected from a group of formula:
—(CH₂)_{g}—,

wherein g is 2-5. R¹³ is selected from the group consisting of H, alkyl and aryl. R¹⁴ is selected from the group consisting of H, alkyl, aryl, and alkoxyalkyl.

In some embodiments, the present invention provides methods of treating metal poisoning (metal detoxification), comprising: a) providing a subject with metal poisoning, and a composition comprising a crowned dithiocarbamate, and b) administering the composition (or any other suitable composition described herein) to the subject. The crowned dithiocarbamate comprises a crown ether-containing group of formula [(CH₂)ₐ—O]_{b}—(CH₂)_{c}, wherein a is at least 2, b is at least 3, and c is at least 2.

In some embodiments, the present invention provides methods of making the compositions embodying features of the present invention comprising reacting pertechnetate with (1) a nitrido donor; (2) a reducing agent; (3) and a crowned DTC chelator. In some embodiments, the nitrido donor is succinyl dihydride, and the reducing agent is stannous chloride.

In some embodiments, the present invention provides kits for the preparation of a radiopharmaceutical embodying features of the present invention comprising: a) a first container (e.g., a bottle) containing a nitrido donor, b) a second container (e.g., a bottle) containing a stannous chloride and a chelating agent able to stabilize the tin cation, and c) a third container (e.g., a bottle) containing a crowned DTC chelator as described herein. In some embodiments, the present invention provides kits for the preparation of a radiopharmaceutical embodying features of the present invention, comprising: a) a first container containing succinyl dihydride, a stannous chloride and a chelating agent able to stabilize the tin cation, and b) a second container containing a crowned DTC chelator as described herein. In some embodiments, the kits comprise: a) a first container containing succinyl dihydride, stannous chloride and 1,2-diaminopropane-N,N,N′,N′-tetraacetic acid or a salt thereof, and b) a second container containing a crowned DTC chelator as described herein.

In some embodiments, the present invention provides kits comprising: a) one or more of compounds or compositions in accordance with the present invention; and b) instructions for using the compounds or compositions for a medical application (e.g. tissue imaging, treating a nitrous oxide related disease, or for metal detoxification). In some embodiments, the compound is in a container (e.g., a vial or bottle). In some embodiments, the instructions are written (e.g., on paper).

In a first series of embodiments, the present invention provides a crowned DTC chelator having a formula (I):

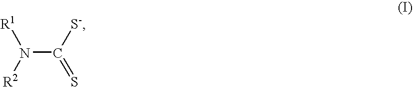

and pharmaceutically acceptable salt thereof, wherein R¹ and R² are the same or different, and are selected from a macrocyclic crown ether-containing group, or R¹ and R² may be taken together to form a macrocycle of formula [(CH₂)ₐ—O]_{b}—(CH₂)_{c}, wherein a is 2-5, b is 3-8, and c is 2-5.

In a second series of embodiments, a is 2 or 3, b is 3-6, and c is 2 or 3 in crowned DTC chelator (I).

In a third series of embodiments, a is 2 and c is 2 in crowned DTC chelator (I).

In a fourth series of embodiments, crowned DTC chelator (I) comprises

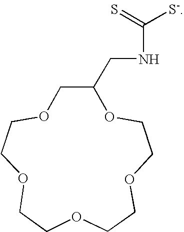

In a fifth series of embodiments, crowned DTC chelator (I) comprises

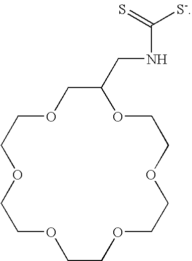

In a sixth series of embodiments, crowned DTC chelator (I) comprises

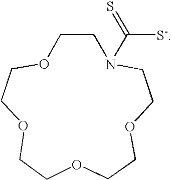

In a seventh series of embodiments, crowned DTC chelator (I) comprises

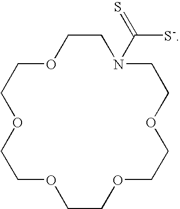

In an eighth series of embodiments, crowned DTC chelator (I) comprises

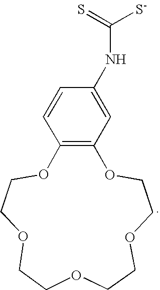

In a ninth series of embodiments, crowned DTC chelator (I) comprises

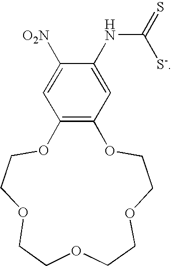

In a tenth series of embodiments, methods for preparing DTC chelators in accordance with the first through ninth series of embodiments described above include reacting an amino crown ether with carbon disulfide in the presence of a base.

In an eleventh series of embodiments, the present invention provides a radiopharmaceutical of formula (M≡N)L¹L² and pharmaceutically acceptable salt thereof, wherein M is a radionuclide selected from the group consisting of ^{99m}Tc, ¹⁸⁶Re, and ¹⁸⁸Re. L¹ and L² are the same or different and comprise a formula (II):

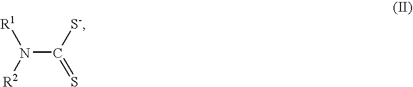

wherein R¹ and R² are the same or different, and are selected from a macrocyclic crown ether-containing group, or R¹ and R² may be taken together to form a macrocycle of the formula [(CH₂)ₐ—O]_{b}—(CH₂)_{c}, wherein a is 2-5, b is 3-8, and c is 2-5.

In a twelfth series of embodiments, M is ^{99m}Tc, a is 2 or 3, b is 3-6, and c is 2 or 3 in radiopharmaceutical (II).

In a thirteenth series of embodiments, a is 2 and c is 2 in radiopharmaceutical (II).

In a fourteenth series of embodiments, radiopharmaceutical (II) comprises:

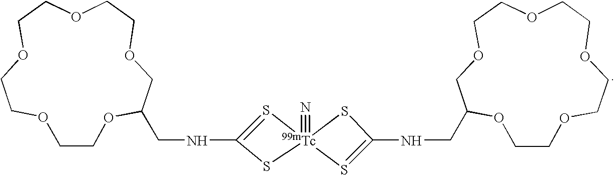

In a fifteenth series of embodiments, radiopharmaceutical (II) comprises:

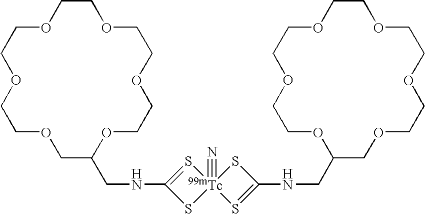

In a sixteenth series of embodiments, radiopharmaceutical (II) comprises:

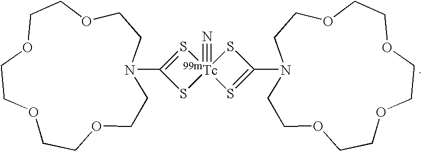

In a seventeenth series of embodiments, radiopharmaceutical (II) comprises:

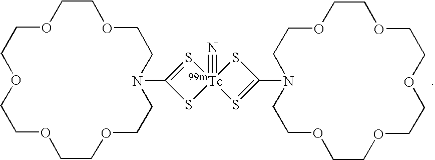

In an eighteenth series of embodiments, radiopharmaceutical (II) comprises:

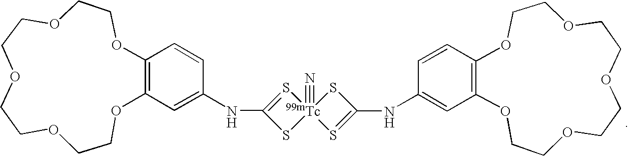

In a nineteenth series of embodiments, radiopharmaceutical (II) comprises:

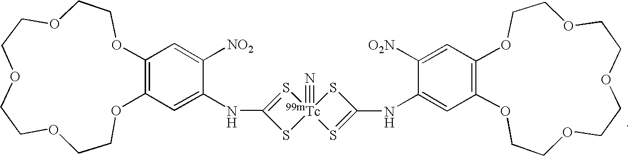

In a twentieth series of embodiments, novel radiopharmaceutical compositions contain a metal chelate according to one of the eleventh through nineteenth series of embodiments described above.

In a twenty-first series of embodiments, methods for preparing radiopharmaceuticals according to the eleventh through nineteenth series of embodiments described above include reacting pertechnetate with (1) a nitrido donor; (2) a reducing agent; (3) and a crowned DTC chelator according to the first through nineteenth series of embodiments described above.

In a twenty-second series of embodiments, in a method according to the twenty-first series of embodiments described above, the nitrido donor is succinyl dihydride, and the reducing agent is stannous chloride.

In a twenty-third series of embodiments, kits for the preparation of a radiopharmaceutical according to the tenth through nineteenth series of embodiments described above include a nitrido donor (e.g., contained in a first container), stannous chloride and a chelating agent able to stabilize the tin cation (e.g., contained in a second container), and a crowned DTC chelator according to the first through ninth series of embodiments described above (e.g., contained in a third container).

In a twenty-fourth series of embodiments, kits for the preparation of a radiopharmaceutical according to the twenty-third series of embodiments described above include a first container containing succinyl dihydride, stannous chloride and a chelating agent able to stabilize the tin cation, and a second container containing a crowned DTC chelator according to the first through ninth series of embodiments described above.

In a twenty-fifth series of embodiments, kits for the preparation of a radiopharmaceutical according to the twenty-fourth series of embodiments described above include a first container containing succinyl dihydride, stannous chloride and 1,2-diaminopropane-N,N,N′,N′-tetraacetic acid or a salt thereof, and a second container containing a crowned DTC chelator according to the fourth through ninth series of embodiments described above.

In a twenty-sixth series of embodiments, methods for radioimaging a mammal embodying features of the present invention include (i) administering to the mammal an effective amount of a radiopharmaceutical according to the eleventh through twenties series of embodiments described above, and (ii) scanning the mammal using a radioimaging device.

In a twenty-seventh series of embodiments, methods for visualizing sites of myocardial disease in a mammal embodying features of the present invention include (i) administering to the mammal an effective amount of a radiopharmaceutical according to the eleventh through twentieth series of embodiments described above, and (ii) scanning the mammal using a radioimaging device.

In a twenty-eighth series of embodiments, methods of diagnosing a myocardial disease in a mammal embodying features of the present invention include (i) administering to the mammal a radiopharmaceutical according to the eleventh through twentieth series of embodiments described above, and (ii) imaging the mammal.

In a twenty-ninth series of embodiments, the present invention provides a novel radiopharmaceutical of formula (III)

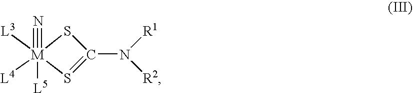

and pharmaceutically acceptable salt thereof, wherein M is a radionuclide selected from the group consisting of ^{99m}Tc, ¹⁸⁶Re, and ¹⁸⁸Re. R¹ and R² are the same or different, and are selected from a macrocyclic crown ether-containing group, or R¹ and R² may be taken together to form a macrocycle of formula [(CH₂)ₐ—O]_{b}—(CH₂)_{c}, wherein a is 2-5, b is 3-8, and c is 2-5. L³, L⁴ and L⁵ are the same or different, and are selected from an isonitrile of formula:

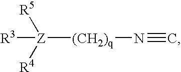

wherein q is 0-3 and Z is carbon or silicon. R³, R⁴ and R⁵ are the same or different, and are selected from the group consisting of H, C₁-C₁₀ alkyl substituted with 0-5 R⁶, aryl substituted with 0-5 R⁶, heteroaryl substituted with 0-5 R⁶, and macrocyclic crown ether containing 2-8 ether-oxygen atoms. R⁶ is independently selected from the group consisting of H, OH, OR⁷, C(═O)OR⁷, C(═O)NR⁸R⁹, PO(OR⁸)₂, PO(NR⁸R⁹)₂ and SO₂R⁷. R⁷, R⁸ and R⁹ are the same or different, and are selected from the group consisting of H, alkyl, aryl, and heteroaryl, or R⁸ and R⁹ may be taken together to form a macrocyclic crown ether containing 2-8 ether-oxygen atoms. Alternatively L³, L⁴ and L⁵ may be taken together to form a tripodal chelator of formula:

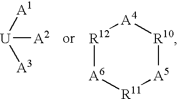

wherein U is selected from the group consisting of R¹³B, CR¹³, and P(═O). A¹, A² and A³ are the same or different, and are imine-N containing heterocycles. A⁴, A¹ and A⁶ are the same or different, and are selected from the group consisting of NR¹⁴, PR¹⁴, S, and O. R¹⁰, R¹¹ and R¹² are the same or different, and are selected from a group of formula:
—(CH₂)_{g}—,

wherein g is 2-5. R¹³ is selected from the group consisting of H, alkyl and aryl group. R¹⁴ is selected from the group consisting of H, alkyl, aryl, and alkoxyalkyl.

In a thirtieth series of embodiments, M is ^{99m}Tc, a is 2 or 3, b is 3-6, c is 2 or 3, q is 1, and Z is carbon in radiopharmaceutical (III). R³, R⁴ and R⁵ are the same or different, and are selected from the group consisting of H, C₁-C₅ alkyl substituted with a R⁶, aryl substituted with a R⁶, heteroaryl substituted with a R⁶, and macrocyclic crown ether containing 3-6 ether-oxygen atoms. R⁶ is selected from the group consisting of OR⁸, C(═O)OR⁸, C(═O)NR⁸R⁹, and PO(NR⁸R⁹)₂. R⁷, R⁸ and R⁹ are the same or different, and are selected from the group consisting of H, alkyl, aryl, and heteroaryl, or R⁸ and R⁹ may be taken together to form a macrocyclic crown ether containing 3-6 ether-oxygen atoms. A¹, A² and A³ are selected from the group consisting of imidazolyl, pyrazolyl, oxazolinyl, methimazolyl, and pyridyl. A⁴, A⁵ and A⁶ are selected from the group consisting of NR¹⁰, PR¹⁰, and S. The subscript g is 2 or 3. R¹³ is selected from the group consisting of H, methyl, and phenyl. R¹⁴ is selected from the group consisting of ethyloxyethyl, ethoxylpropyl, methyoxyethyl, and methoxypropyl.

In a thirty-first series of embodiments, a is 2 and c is 2 in radiopharmaceutical (III). R³, R⁴ and R⁵ are the same or different and are selected from the group consisting of H, C₁-C₅ alkyl, phenyl, and macrocyclic crown ether containing 3-6 ether-oxygen atoms. R⁷, R⁸ and R⁹ can be the same or different, and are selected from the group consisting of H, alkyl, and phenyl, or R⁸ and R⁹ may be taken together to form a macrocyclic crown ether containing 3-6 ether-oxygen atoms. L³, L⁴ and L⁵ are taken together to form a tripodal chelator having a formula selected from the group consisting of:

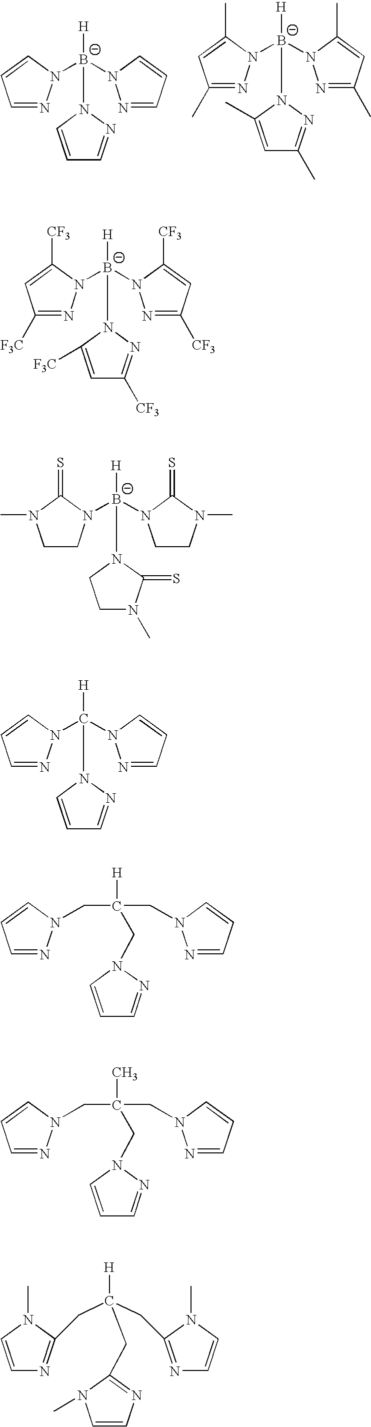

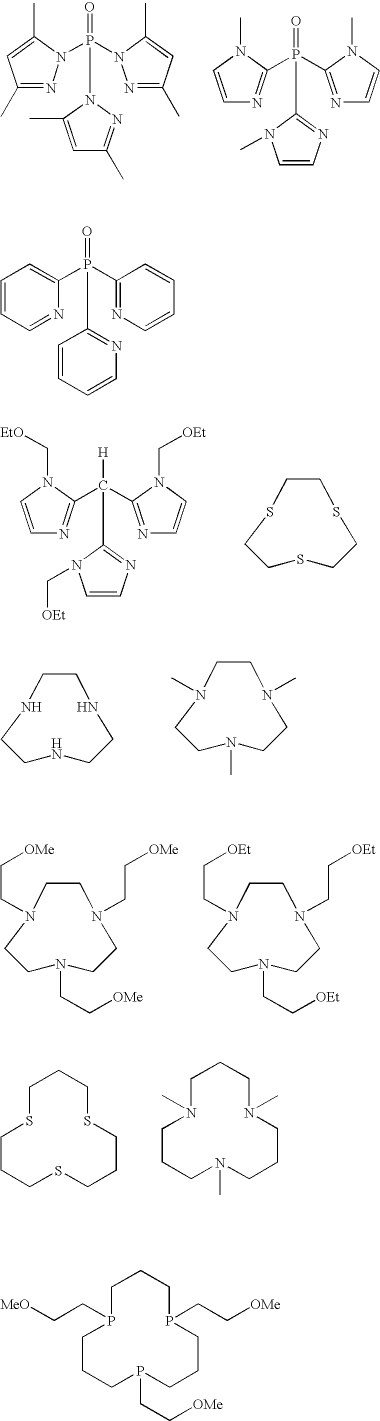

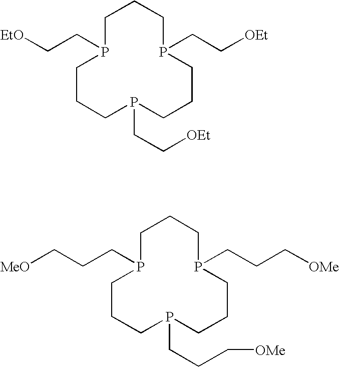

In a thirty-second series of embodiments, L³, L⁴ and L⁵ are the same in radiopharmaceutical (III) and are selected from any one of following macrocyclic crown ether-containing isonitriles:

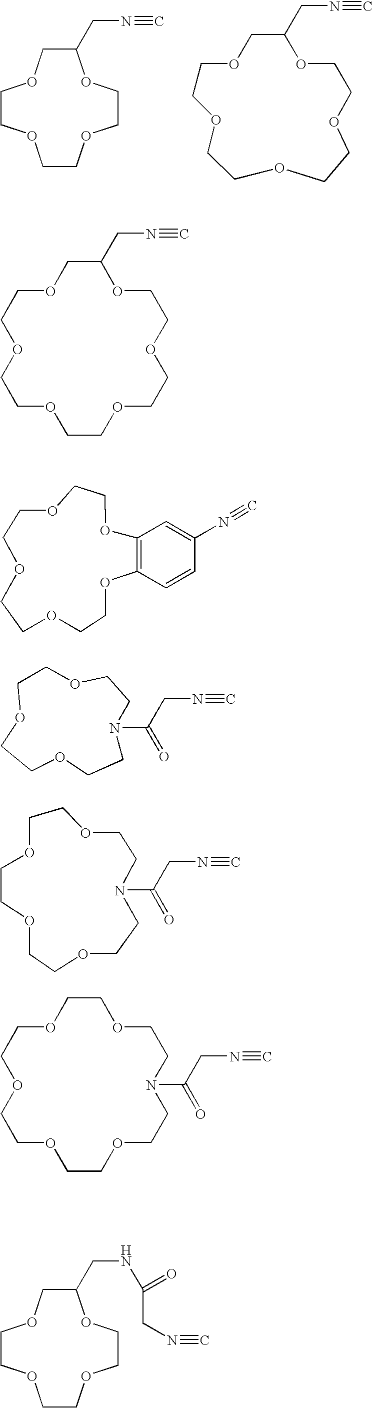

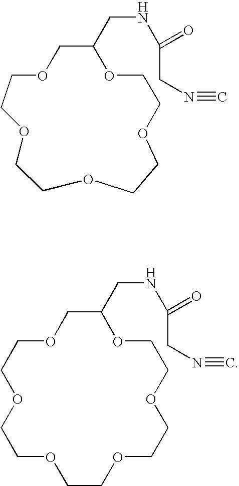

In a thirty-third series of embodiments, the dithiocarbamate chelator in radiopharmaceutical (III) is selected from any one of the following crowned DTCs:

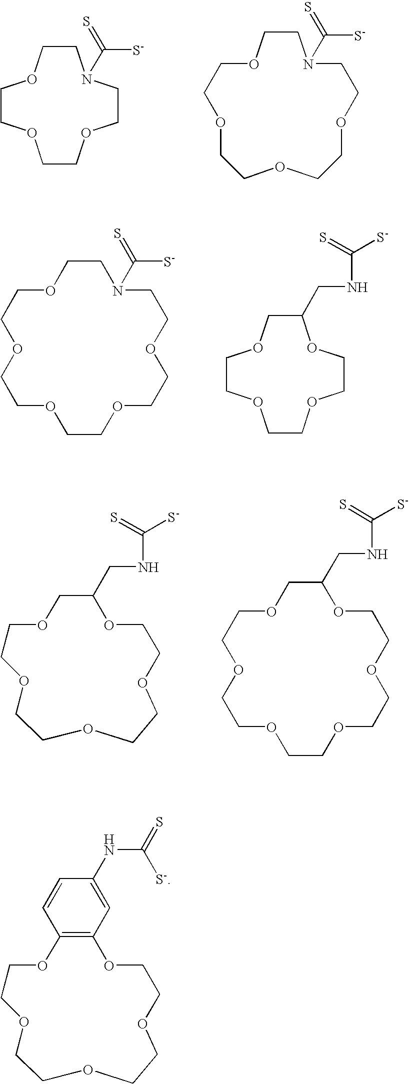

In a thirty-fourth series of embodiments, novel radiopharmaceutical compositions embodying features of the present invention include a metal chelate according to the twenty-ninth through thirty-third series of embodiments described above.

In a thirty-fifth series of embodiments, methods for the preparation of a radiopharmaceutical according to the twenty-ninth through thirty-third series of embodiments described above include reacting pertechnetate with (1) a nitrido donor; (2) a reducing agent; (3) an organic isonitrile ligand or tripodal chelator according to the twenty-ninth through thirty-third series of embodiments described above, and a crowned DTC chelator according to the first through ninth series of embodiments described above.

In a thirty-sixth series of embodiments, in a method according to the thirty-fifth series of embodiments described above, the nitrido donor is succinyl dihydride, and the reducing agent is stannous chloride.

In a thirty-seventh series of embodiments, kits for the preparation of a radiopharmaceutical according to twenty-ninth through thirty-third series of embodiments described above includes: a first container containing a nitrido donor, a stannous chloride and a chelating agent able to stabilize the tin cation; a second container containing an organic isonitrile ligand or a tripodal chelator according to the twenty-ninth through thirty-third series of embodiments described above; and a third container containing a crowned DTC chelator according to the first through ninth series of embodiments described above.

In a thirty-eighth series of embodiments, kits for the preparation of a radiopharmaceutical according to the thirty-seventh series of embodiments described above include: a first container containing a succinyl dihydride, a stannous chloride and 1,2-diaminopropane-N,N,N′,N′-tetraacetic acid or a salt thereof, and a second container containing an organic isonitrile ligand or a tripodal chelator according to the twenty-ninth through thirty-third series of embodiments described above, and a crowned DTC chelator according to the first through ninth series of embodiments described above.

In a thirty-ninth series of embodiments, kits for the preparation of a radiopharmaceutical product according to the thirty-sixth series of embodiments described above includes: a first container containing a succinyl dihydride, a stannous chloride and 1,2-diaminopropane-N,N,N′,N′-tetraacetic acid or a salt thereof, and a second container containing an organic isonitrile ligand according to the twenty-ninth through thirty-third series of embodiments described above, and a crowned DTC chelator according to fourth through ninth series of embodiments described above.

In a fortieth series of embodiments, methods for radioimaging a mammal include (i) administering to the mammal an effective amount of a radiopharmaceutical according to the twenty-ninth through thirty-third series of embodiments described above, and (ii) scanning the mammal using a radioimaging device.

In a forty-first series of embodiments, methods for visualizing sites of myocardial disease in a mammal by radioimaging, include (i) administering to the mammal an effective amount of a radiopharmaceutical according to the twenty-ninth through thirty-third series of embodiments described above, and (ii) scanning the mammal using a radioimaging device.

In a forty-second series of embodiments, methods of diagnosing a myocardial disease in a mammal include (i) administering to the mammal a radiopharmaceutical composition according to embodiments according to the twenty-ninth through thirty-third series of embodiments described above, and (ii) imaging the mammal.

In a forty-third series of embodiments, the present invention provides a pharmaceutical of formula (IV):

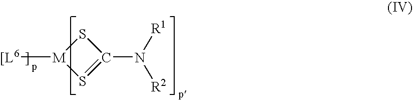

and pharmaceutically acceptable salt thereof, wherein M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II). The subscripts p and p′ are integers and are independently selected from 0-2. R¹ and R² are the same or different, and are selected from a macrocyclic crown ether-containing group, or R¹ and R² may be taken together to form a macrocycle of formula [(CH₂)ₐ—O]_{b}—(CH₂)_{c}, wherein a is 2-5, b is 3-8, and c is 2-5. L⁶ is a tripodal chelator to complete the coordination sphere of the transition metal, and is selected from a compound of formula:

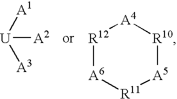

wherein U is selected from the group consisting of R¹³B, CR¹³, and P(═O). A¹, A² and A³ are the same or different, and are imine-N containing heterocycles. A⁴, A⁵ and A⁶ are selected from the group consisting of NR¹⁰, PR¹⁰, and S. R¹⁰, R¹¹ and R¹² are the same or different, and are selected from a group of formula:
—(CH₂)_{g}—,

wherein g is 2-5. R¹³ is selected from the group consisting of H, alkyl, and aryl. R¹⁴ is selected from the group consisting of H, alkyl, aryl, and alkoxyalkyl.

In a forty-fourth series of embodiments, in pharmaceutical (IV), M is Ru(III), p is 1, and p′ is 2. R¹ and R² are the same or different, and are selected from a macrocyclic crown ether-containing group, or R¹ and R² may be taken together to form a macrocycle of formula [(CH₂)ₐ—O]_{b}—(CH₂)_{c}, wherein a is 2 or 3, b is 3-6, and c is 2 or 3. A¹, A² and A³ are selected from the group consisting of imidazolyl, pyrazolyl, oxazolinyl, methimazolyl, and pyridyl. A⁴, A⁵ and A⁶ are selected from the group consisting of NR¹⁰, PR¹⁰, and S. The subscript g is 2 or 3. R¹³ is selected from the group consisting of H, methyl, and phenyl. R¹⁴ is selected from the group consisting of ethyloxyethyl, ethoxylpropyl, methyoxyethyl, and methoxypropyl. R¹⁴ is selected from the group consisting of H, alkyl, aryl, and alkoxyalkyl.

In a forty-fifth series of embodiments, in pharmaceutical (IV), a is 2, c is 2, and L⁶ is selected from any one of the following tripodal chelators:

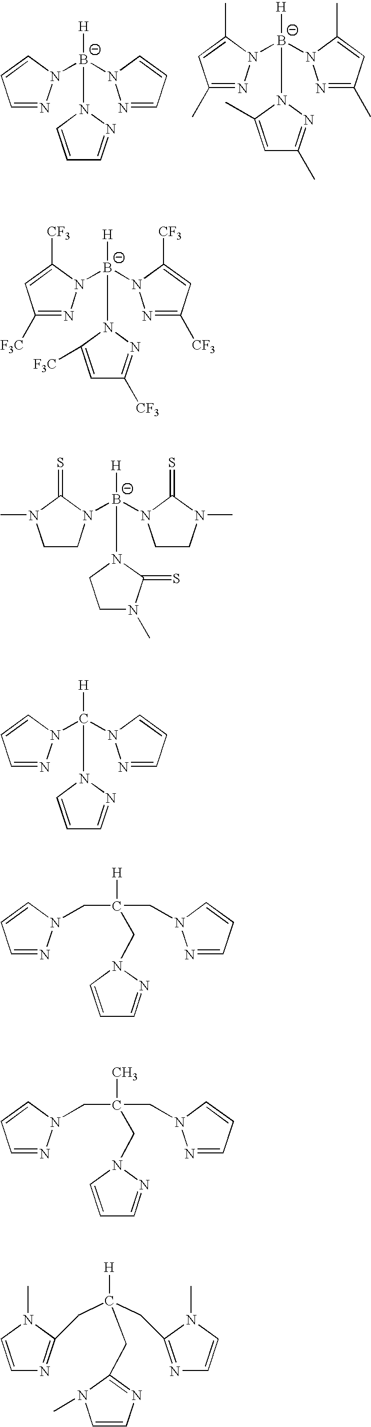

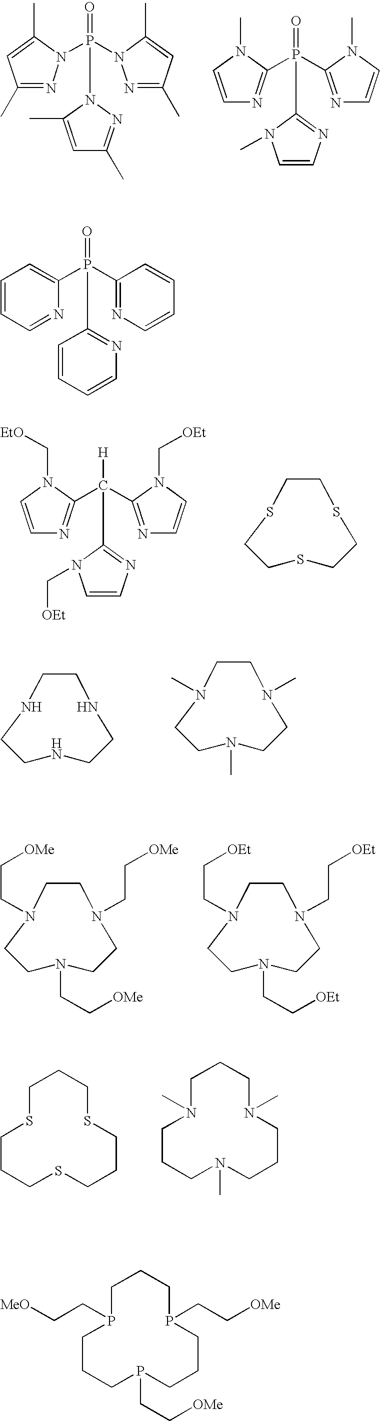

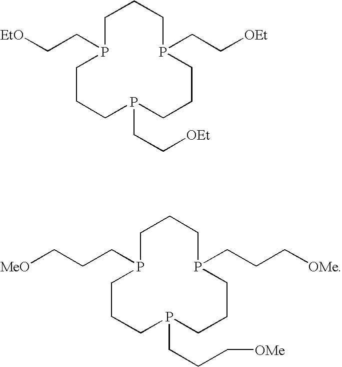

In a forty-sixth series of embodiments, the dithiocarbamate chelator in pharmaceutical (IV) is selected from any one of the following crowned DTCs:

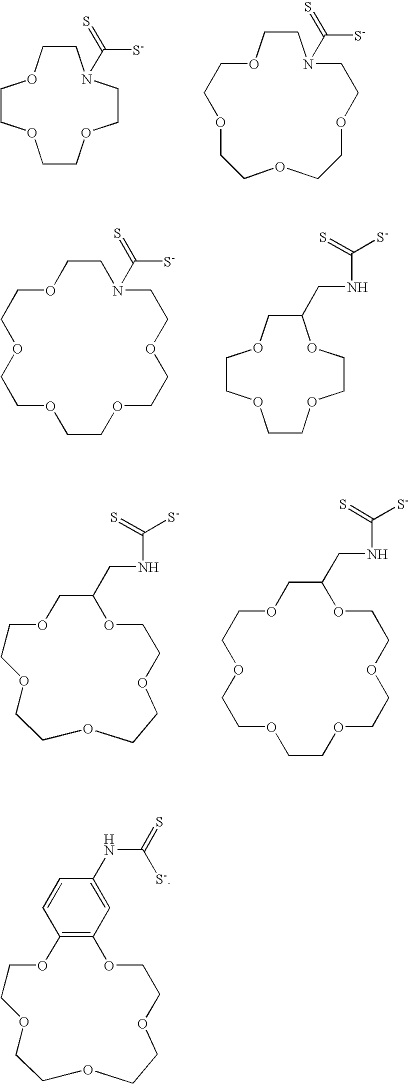

Diagnostic and Therapeutic Uses

Crowned dithiocarbamate metal complexes embodying features of the present invention may be used as pharmaceutical agents to diagnose or treat disease.

Radiopharmaceutical Compositions

In some embodiments, the crowned dithiocarbamate metal complexes embodying features of the present invention are neutral and cationic radioactive metal-nitrido complexes of crowned dithiocarbamates and are used as radiopharmaceuticals. Radiopharmaceuticals are drugs containing a radionuclide, and are used routinely in nuclear medicine for the diagnosis or therapeutic treatment of various diseases. They are mostly small organic or inorganic compounds with definite compositions. Radiopharmaceuticals form the chemical basis for nuclear medicine, a group of techniques used for diagnosis and therapeutic treatment of various diseases. The in vivo diagnostic information is obtained by intravenously injecting the radiopharmaceutical and determining its biodistribution using a gamma camera. The biodistribution of the radiopharmaceutical depends on the physical and chemical properties of the radiopharmaceutical and may be used to obtain information about the presence, progression, and state of disease. The radioactive metal-nitrido complexes of crowned dithiocarbamates embodying features of the present invention may be used as radiopharmaceuticals.

The radionuclide for a diagnostic radiopharmaceutical is often a gamma-emitting isotope for scintigraphic imaging or a positron-emitting isotope for positron emission tomography (PET). The choice of radionuclide depends largely on the physical and nuclear properties (e.g., half-life and γ-energy), availability, and cost. Nearly 80% of all radiopharmaceuticals used in nuclear medicine are ^{99m}Tc-labeled compounds. The 6 hour half-life is long enough to allow a radiochemist to carry out radiopharmaceutical synthesis and for nuclear medicine practitioners to collect useful images. At the same time, it is short enough to permit the administration of millicurie amounts of ^{99m}Tc radioactivity without providing significant radiation dosages to the patient. The monochromatic 140 KeV photons are readily collimated to give images of superior spatial resolution. Furthermore, ^{99m}Tc is readily available from commercial ⁹⁹Mo—^{99m}Tc generators at low cost.

In some preferred embodiments, the metallic radionuclide is selected from the group consisting of ^{99m}Tc, ¹⁸⁶Re and ¹⁸⁸Re. For diagnostic purposes, ^{99m}Tc is a preferred isotope. Its 6 hour half-life and 140 keV gamma ray emission energy are almost ideal for gamma scintigraphy using equipment and procedures well established in the art. The rhenium isotopes also have gamma ray emission energies that are compatible with gamma scintigraphy; however, they also emit high energy beta particles that are more damaging to living tissues. These beta particle emissions can be utilized for therapeutic purposes, for example, cancer radiotherapy. The related chemistry, medical applications, and radiolabeling with ^{186/188}Re by direct and indirect methods have been reviewed (Fritzberg et al. Pharmaceutical Res. 1988, 5, 325; Liu et al. Bioconjugate Chem. 1997, 8, 621; Dilworth, J. R. and Parrott, S. J. Chem. Soc. Rev. 1998, 27, 43).

^{99m}Tc is produced from a parent radionuclide, ⁹⁹Mo, a fission product with a half-life of 2.78 days. In a ⁹⁹Mo—^{99m}Tc generator, [⁹⁹Mo]molybdate is absorbed to an alumina column and ^{99m}Tc is formed by decay of ⁹⁹Mo. The ^{99m}Tc in the form of [^{99m}Tc]pertechnetate is eluted from the column with saline. The ^{99m}Tc produced by the generator is never carrier-free because fifteen percent of ⁹⁹Mo decays directly to the long-lived isotope ⁹⁹Tc (t_{1/2}=2.13×10⁵ y), which is also the single decay product of ^{99m}Tc. The specific activity of eluted ^{99m}Tc is very high and is dependent upon the prior-elution time. In general, the total concentration of technetium (^{99m}Tc and ^{99m}Tc) in the ⁹⁹Mo—^{99m}Tc generator eluent is in the range of 10⁻⁷-10⁻⁶ M.

For the last two decades, PET imaging was only used for academic research, most likely due to the short half-lives of isotopes, availability of generator systems, practicality of isotope production, and transportation and distribution of the radiotracer. The development of outside vendors who can supply PET isotopes to a number of local customers on a unit dose basis, and the adaptability of SPECT cameras for PET imaging should increase the use of this imaging modality (Phelps, M. E. J. Nucl. Med. 2000, 41, 661; Bar-Shalom et al. Seminars Nucl. Med. 2000, 30, 150; and O'Doherty, M. J. Nucl. Med. Commun. 2000, 21, 224). In some embodiments, detection in accordance with the present invention is performed by PET imaging (e.g., using a SPECT camera or similar type camera).

Compared to other imaging modalities, PET has three important technological features, which enable clinicians to measure biochemical or physiological process in vivo. The first feature is its ability to accurately measure the actual 3-D radiotracer distribution, which makes PET similar to autoradiography. The second feature is its ability to rapidly acquire a dynamic set of tomographic images through a volume of tissue. This is unique for PET imaging because no other imaging modality except MRI has such ability. The third feature is the ability to acquire whole body images. It is the combination of these three features with the high specificity of receptor binding of biomolecules that makes PET imaging using radiolabeled biomolecules extremely attractive for nuclear medicine.

^{94m}Tc is a cyclotron-produced isotope with a half-life of 52 min (0.9 h) and a β⁺ energy of 2.47 MeV (72%). It can be obtained from a number of production methods, including ⁹⁴Mo(p, n)/^{94m}Tc (13.5-11 MeV), ^{nat}Nb(³He, 2n)/^{94m}Tc (18-10 MeV), ⁹²Mo(α, pn)/^{94m}Tc (26-18 MeV). The quantitative superiority of PET permits modeling of radiotracer kinetics and dosimetry measurements. The successful preparation of ^{94m}Tc in the pertechnetate form allows the use of the same commercially available kit for ^{99m}Tc radiopharmaceuticals to prepare the ^{94m}Tc analogs. The use of dual isotopes ^{99m}Tc/^{94m}Tc (SPECT/PET) may provide much better imaging quality of diseased tissue. The integration of PET and SPECT radiotracer development would pave the way for better exploitation of the current strengths of the two imaging modalities, and will be extremely important for both the oncology and cardiology applications of radiopharmaceuticals such as ^{99m}Tc-Sestamibi and ^{99m}Tc-Tetrofosamin. As such, the use of both ^{99m}Tc/^{94m}Tc in the radioactive metal-nitrido complexes of crowned dithiocarbamates embodying features of the present invention is preferred in certain embodiments.

Rhenium shares similar coordination chemistry with technetium due to their periodic relationship. Rhenium has two isotopes (¹⁸⁶Re and ¹⁸⁸Re) that are useful for radiotherapy. ¹⁸⁶Re has a half-life of 3.68 days with a β⁻-emission (Emax=1.07 MeV, 91% abundance) and a gamma-photon (E=137 keV, 9% abundance) which should allow imaging during therapy. ¹⁸⁶Re is a reactor-produced radionuclide, and is obtained by the irradiation of ¹⁸⁵Re with neutrons (¹⁸⁵Re(n, γ)¹⁸⁶Re). The yield of ¹⁸⁶Re depends on the amount of Re target, the energy of the neutrons available, and the neutron reflux. The specific activity is low or medium, but a carrier-free product is not possible. As such, in certain embodiments, the radioactive metal-nitrido complexes of crowned dithiocarbamates in accordance with the present invention employ ¹⁸⁶Re and ¹⁸⁸Re as the radioactive metal.

¹⁸⁸Re has a half-life of 16.98 h with a high-energy β-emission (Emax=2.12 MeV, 85% abundance) and 155 keV gamma photons (15% abundance). ¹⁸⁸Re can be prepared either from the nuclear reaction (¹⁸⁷Re(n, γ)¹⁸⁸Re) or from the ¹⁸⁸W—¹⁸⁶Re generator. The generator-produced ¹⁸⁸Re is carrier-free and has very high specific activity. The major advantage of using ¹⁸⁸Re in therapeutic nuclear medicine is the inexpensive and readily available ¹⁸⁸W—¹⁸⁶Re generator, which has a very long and useful shelf-life.

There are many Tc cores for the routine synthesis of ^{99m}Tc radiopharmaceuticals. The [Tc═O]³⁺ core is stable in the presence of a strong chelator in aqueous media. It is the most frequently used Tc core for ^{99m}Tc radiopharmaceuticals. The [Tc═O]³⁺ core forms square pyramidal Tc-oxo chelates with tetradentate chelators, including N₄ propylene amine oxime (PnAO), N₃S triamidethiols, N₂S₂ diamidedithiols (DADS), N₂S₂ monoamidemonoaminedithiols (MAMA), and N₂S₂ diaminedithiols (DADT). The [Tc≡N]²⁺ core is isoelectronic with the [Tc═O]³⁺ core. The nitrido ligand is a powerful π-electron donor and shows a high capacity to stabilize the Tc(V) oxidation state. The [Tc≡N]²⁺ core forms Tc(V) nitrido complexes with a variety of chelators. Various chelators have been used for the preparation of ^{99m}Tc radiopharmaceuticals. ^{99m}Tc-labeling techniques have been extensively reviewed (See, e.g., Hom, R. K. and Katzenellenbogen, J. A. Nucl. Med. Biol. 1997, 24, 485; Dewanjee, M. K. Semin. Nucl. Med. 1990, 20, 5; and Jurisson et al Chem. Rev. 1993, 93, 1137).

International PCT application no. WO 90/06137 describes a series of technetium-nitrido chelates of dithiocarbamates, including dimethyldithiocarbamate, di-n-propyl dithiocarbamate, N-ethyl-N-(2-ethyoxyethyl)dithiocarbamate. International PCT application nos. WO 89/08657, WO 92/00982, and WO 93/01839 describe processes for producing technetium nitrido complexes, which include reacting a polyphosphine as a reducing agent for the technetium oxide, then reacting with a nitride salt of a metal or ammonium ion. Since Tc-nitrido core has four to five coordination sites for various ligands or chelators, the choice of chelator is important for the solution stability and the number of radioactive species formed during ligand exchange reaction.

U.S. Pat. Nos. 5,288,476 and 6,071,492 describe cardiac tropism radiopharmaceutical products incorporating a nitride complex of a transition metal, which have a rapid myocardial clearance. Substituents on the dithiocarbamate N atoms are selected from a branched alkyl group having one or more ether functions, a tetrahydrofurfuryl or ether group, and a tetrahydrofurfuryl or dioxaspiro or dialyoxy piperidino group. Because of these ether-containing groups, the ^{99m}Tc complexes of dithiocarbamate show rapid clearance from the liver and lungs, resulting in high heart/liver and heart/lung ratios. These references do not teach or suggest the formation of macrocyclic crown ethers from the two substituents on the dithiocarbamate-N atom, nor the use of crowned DTCs for the preparation of ^{99m}Tc-nitrido complexes as described herein.

International PCT application no. WO 98/27100 describes ^{99m}Tc chelate radiopharmaceuticals including ^{99m}Tc-nitrido and two different bidentate ligands coordinated therewith. Although the bisphosphine was originally proposed as a bidentate chelator, structural studies have clearly demonstrated that the bisphosphine chelator is actually tridentate to form the six-coordinate Tc-nitrido complex with a bidentate chelator, such as dithiocarbamate, as coligand.

International PCT application no. WO 02/09771 describes a new class of asymmetric cationic ^{99m}Tc-nitrido complexes, which contain a [^{99m}Tc≡N]²⁺ core, a tridentate PXP bisphosphine, and a DTC chelator. It was found that these cationic ^{99m}Tc-nitrido complexes are rapidly extracted by the myocardium of rats, and retained in the heart for a long time. The lung uptake became negligible at 5 min post-injection, and liver washout was also very fast. Substituents on the phosphine P, secondary amine N, and dithiocarbamate N atoms are selected from alkyl, aryl, alkoxy, or alkoxyalkyl groups. This reference does not teach or suggest the use of crowned DTCs for the preparation of ^{99m}Tc-nitrido complexes.

Ischemia-related diseases, particularly coronary artery disease (CAD), account for the majority of death in Western countries. Myocardial ischemia is a serious condition and the delay in reperfusion of the ischemic tissues can be life threatening. This is particularly true in the aged population. Rapid and accurate early detection of myocardial ischemia is highly desirable so that various therapeutic regiments can be given before irreversible myocardial damage occurs. In this regard, the compositions of the present invention are preferably used for myocardial perfusion imaging.

Myocardial perfusion imaging with radiotracers is an integral component of the clinical evaluation of patients with known or suspected coronary artery disease (CAD) in current clinical practice (See, e.g., Acmpa, W. et al. J. Nucl. Cardiol. 2000, 7, 701; Berman, D. et al. Semin. Nucl. Med. 1999, 29, 280; and Dilsizian, V. J. Nucl. Cardiol. 2000, 7, 180). The introduction of thallium-201 (²⁰¹Tl) in the mid 1970s was the turning point in the widespread clinical use of myocardial perfusion imaging, and had a profound impact on diagnostic evaluation, risk stratification, and therapeutic decision-making in patients with CAD over the last two decades. However, ²⁰¹Tl has its limitations. The vulnerability of ²⁰¹Tl to attenuation artifacts caused by the relatively lower energy emitted photons and lower count rate caused by the dose constraints may result in poor or suboptimal images in a significant proportion of studies. In addition, ²⁰¹Tl images should be taken soon after injection mainly due to the dynamic nature of its distribution and redistribution dynamics, and may not be suitable for situations where immediate imaging may not be possible (for example, patients with acute myocardial infarction).

Compared to ²⁰¹Tl, ^{99m}Tc yields relatively high-energy photons and can be used at much higher doses. The use of ^{99m}Tc also allows the simultaneous assessment of myocardial perfusion and cardiac function in a single study (Kapur, A. et al. Eur. J. Nucl. Med. 2002, 29, 1608). Because of its ideal nuclear properties (short half-life and γ-energy) and its diverse coordination chemistry, ^{99m}Tc has been the isotope of choice for the development of myocardial perfusion imaging agents.

^{99m}TcN-Noet is a member of the neutral ^{99m}Tc-nitrido complexes, which are characterized by the presence of the ^{99m}Tc≡N triple bond and two N-alkyl dithiocarbamate ligands. Duatti and coworkers first reported the synthesis of ^{99m}Tc-nitrido complexes with various chelators and their use as heart imaging agents (Marchi, A. et al. J. Chem. Soc. Dalton Trans. 1990, 1743; Duatti, A. et al. J. Chem. Soc., Dalton Trans. 1990, 3729; Marchi, A et al. Inorg. Chem. 1990, 29, 2091). Biodistribution studies demonstrated that these neutral ^{99m}Tc-nitrido complexes are localized in the myocardium of rats, dogs, and primates. The high quality of the myocardial images obtained in dogs and monkeys demonstrates that ^{99m}TcN-Noet has the most favorable distribution properties. ^{99m}TcN-Noet is currently in phase III clinical trials in Europe.

One aspect of the present invention relates to neutral ^{99m}Tc-nitrido complexes as new radiopharmaceuticals for myocardial imaging. The ^{99m}Tc-nitrido complexes described herein are expected to have a first-pass extraction comparable to or better than that of ^{99m}Tc—N-Noet due to their structural similarity. The presence of crown ether groups in compounds embodying features of the present invention should allow a faster clearance of ^{99m}Tc-nitrido complexes from the liver and lungs, and better heart/liver and heart/lung ratios.

Another aspect of the present invention relates to cationic ^{99m}Tc-nitrido complexes containing two different chelators, one of which is a crowned DTC, and to their use as new radiopharmaceuticals for imaging (e.g., myocardial perfusion imaging). These cationic ^{99m}Tc-nitrido complexes are expected to have a higher heart uptake and longer myocardial retention than ^{99m}Tc—N-Noet due to the cationic character and possible interactions between the crown ether moiety and intracellular K⁺. The presence of crown ether groups also results in a faster renal clearance with less hepatobiliary uptake and gastrointestinal retention than ^{99m}Tc—N-Noet, ^{99m}Tc-Sestamibi and ^{99m}Tc-Tetrofosmin due to increased hydrophilicity.

Zhang and coworkers recently reported the use of neutral ^{99m}Tc-nitrido complexes as brain imaging agents (Zhang, J. et al Nucl. Med. Biol. 2002, 29, 665; Zhang, J. et al. Appl. Radiat. Isot. 2002, 56, 857; Zhang, J. et al. Appl. Radiat. Isot. 2001, 54, 745; and Zhang, J. and Wang, X. Appl. Radiat. Isot. 2001, 55, 453). In these complexes, substituents on the dithiocarbamate N atom are simple alkyl or cycloalkyl groups. Compared to cationic agents, the clearance of ^{99m}TcN-Noet from blood is significantly slower. ^{99m}TcN-Noet also shows high initial pulmonary uptake and prolonged liver retention. The combination of slow blood clearance and pulmonary uptake imposes a significant challenge for optimal myocardial perfusion imaging. In contrast, compositions in accordance with the present invention have high uptake in the heart and reduced liver retention.

Recently, Duatti and coworkers (Boschi, A. et al. Nucl. Med. Commun. 2002, 23, 689; Bolzati, C. et al. J. Am. Chem. Soc. 2002, 124, 11468) reported a new class of asymmetric cationic ^{99m}Tc-nitrido complexes, which contain a [^{99m}Tc≡N]²⁺ core, a tridentate PXP bisphosphine, and a DTC chelator, and their biological evaluation as radiopharmaceuticals for heart imaging. It was found that these cationic ^{99m}Tc-nitrido complexes are rapidly extracted by the myocardium of rats and retained in the heart for a long time. The lung uptake became negligible at 5 min post-injection, and liver washout was also very fast. Heart/liver ratios were increased exponentially with time, and the liver activity was almost completely eliminated into the intestine at 60 min post-injection. The heart/liver ratios were ˜10 times higher than those of ^{99m}Tc-Sestamibi and ^{99m}Tc-Tetrofosmin in the same animal model.

It should be noted that the heart uptake of lipophilic cations is not just limited to ^{99m}Tc complexes. It has been reported that cationic ⁶⁴Cu complexes show high heart uptake (Packard, A. B. Nucl. Med. Biol. 1998, 25, 531; Packard, A. B. Nucl. Med. Biol. 2002, 29, 289). Lipophilic ⁶⁸Ga complex cations have also been found to show a high heart uptake and are useful for evaluation of myocardial perfusion using PET (Tsang, B. W. et al. J. Nucl. Med. 1993, 34, 1127; Tsang, B. W. et al. J. Med. Chem. 1994, 37, 4400). It has also been demonstrated that the 3-methoxy or 3-ethoxy group is very important for high heart uptake for ⁶⁸Ga chelates. Studies on a Q-series of cationic ^{99m}Tc complexes also showed that pendant ether moieties from phosphine ligands could improve the myocardial imaging properties (Lisic, E. C. et al. Nucl. Med. Biol. 1999, 26, 563; Marmion, M. E. et al. Nucl. Med. Biol. 1999, 26, 755).

In addition to their use in cardiology applications, the radioactive metal-nitrido complexes of crowned dithiocarbamates embodying features of the present invention (e.g., ^{99m}Tc complexes) may also be used as radiopharmaceuticals for non-invasive imaging of any type of tissue, including but not limited to tumor MDR1 (multidrug resistance) p-glycoprotein (Pgp) transport function (Sharma, V. and Piwnica-Worms, D. Chem. Rev. 1999, 99: 2545; and Herman, L. et al. J. Med. Chem. 1995, 38, 2955). Various cationic ^{99m}Tc complex radiopharmaceuticals, originally developed for myocardial perfusion imaging, have been shown to be substrates for transport by MDR1 Pgp.

A crown ether containing dithiodicarbamate and its Co(II), Ni(II), Cu(II) and Zn(II) complexes have been synthesized and characterized (Wang, J. H. and Wang, Y. L. Yingyong Huaxue 2002, 19, 295-297; Wang, J.-H. and Zhang, Z. Yingyong Huaxue 1994, 11, 101). It was found that all metal complexes were stable, and that the dithiocarbamate group is bidentate. The crystal structure of cobalt tris[(aza-15-crown-5)dithiocarbamate has also been reported (Granell, G. et al J. Chem. Soc., Dalton Trans. 1990, 605); but no specific applications were described.

Additional Therapeutic Uses

Compounds embodying features of the present invention, in addition to their use as radioimaging agents, may also be used to treat disease. The enhanced profile of metal in medicine in recent years cascades from the ongoing search for new therapeutic agents with unique mechanisms of action (Abrams, M. J. and Orvig, C. Chem. Rev. 1999, 99, 2201; Thompson, K. H. and Orvig, C. Science 2003, 300, 936; Clarke, M. J. Coord. Chem. Rev. 2002, 232, 69-93). The therapeutic uses of ruthenium complexes, in particular, are of interest and have been investigated as immunosuppressive agents (Bastos, C. M. et al. Bioorg. Med. Chem. Lett. 1998, 8, 147), antitumor and anti-metastatic agents (Sava, G. et al. Top. Biol. Inorg. Chem. 1999, 1, 143; Sava, G. et al. Chem.-Biol. Interact. 1995, 95, 109; Sava, G. et al. Top. Biol. Met.-Based Drugs 1995, 2, 221), and nitric oxide (NO) scavengers (Cameron, B. R. Inorg. Chem. 2003, 42, 4102; Chatterjiee, D. et al. Dalton Trans. 2003, 203). The overproduction of NO has been implicated to play a significant role in many disease states such as septic shock (Evans, T. et al. Circ. Schock 1993, 41, 77), rheumatoid arthritis (Wei, et al. Nature 1995, 375, 408; Stefanovic-Racic, M. et al. Arthritis Rheum. 1993, 36, 1036), diabetes (Corbett, J. A. and McDaniel, M. L. Diabetes 1992, 41, 849), asthma (Hamid, Q. et al. Lancet 1993, 342, 1510), and cancer (Gallo, O. et al. J. Natl. Cancer Inst. 1998, 90, 587). Therefore, in certain embodiments, Ru(III) metal complex pharmaceuticals embodying features of the present invention may be beneficial for the treatment of diseases such as septic shock, rheumatoid arthritis, diabetes, asthma, and cancer.

Dithiocarbamates (DTCs) are known heavy metal chelators (Sunderman, F. W. Ann. Clin. Lab. Sci. 1978, 8, 259; Jones, M. M. and Cherian, M. G. Toxicology 1990, 62, 1). DTCs such as diethyldithiocarbamate have been clinically used for the treatment of nickel poisoning, and were used in clinical trials for the treatment of AIDS patients (Reisinger, E. et al. Lancet 1990, 335, 679). DTCs such as pyrrolidine dithiocarbamate are potent inhibitors of nuclear factor kappa B in intact cells (Schreck, R. et al. J. Exp. Med. 1992, 175, 1181). In addition, nuclear factor kappa B has been shown to up-regulate the expression of cell adhesive molecules, including the vascular cell adhesive molecule 1 (VCAM-1) (Lademarco et al. J. Biol. Chem. 1992, 267, 16323). Endothelial expression of VCAM-1 causes the adherence of neutrophils to the endothelium, an early event leading to inflammation and subsequent vascular damage and reduction of blood flow (Oppenheimer, M. N. et al. J. Immunol. 1991, 147, 42207). Therefore, DTCs and their metal complexes would block VCAM-1 expression, thereby avoiding the vascular problems associated with neutrophil adherence to the endothelium.

International PCT application no. WO 01/62085 A1 describes conjugates of dithiocarbamates and pharmacologically active agents for the treatment of inflammatory diseases. United States patent publication no. US 2002/0045573 A1 also describes DTC-containing drugs for therapeutic treatment of such indications as cerebral stroke and other ischemia/reperfusion injuries. In the described agents, the DTC moiety is linked to the surface of a non-immunogenic, non-targeting macromolecule other than an antibody. The metal chelates of a composition comprising a DTC and a non-immunogenic, non-targeting macromolecule can also be used for the same purpose (US 2002/0045573 A1). Recent studies have shown that DTC chelators act either as a direct scavenger of hydroxy radicals (due to their thiol group) or as an iron chelator that inhibits hydroxyl radical production by binding to the iron ions or by both mechanisms (Liu et al. Free Rad. Res. 1996, 24, 461).

Another aspect of the present invention relates to compositions of (e.g., neutral or cationic metal chelates) as nitric oxide (NO) scavengers. The presence of crown ether groups may be used to increase water solubility. Metal chelate-based NO scavengers may be useful as therapeutic pharmaceuticals for the treatment of diseases including but not limited to septic shock, rheumatoid arthritis, diabetes, asthma, and cancer.

In the last decade, nitric oxide has been widely studied because of its essential role in many physiological processes. The overproduction of NO has been implicated to play a significant role in many disease states, such as septic shock (Evans, T. et al. Circ. Schock 1993, 41, 77), rheumatoid arthritis (Wei, et al. Nature 1995, 375, 408; Stefanovic-Racic, M. et al. Arthritis Rheum. 1993, 36, 1036), diabetes (Corbett, J. A. and McDaniel, M. L. Diabetes 1992, 41, 849), asthma (Hamid, Q. et al. Lancet 1993, 342, 1510), and cancer (Gallo, O. et al. J. Natl. Cancer Inst. 1998, 90, 587). Therefore, compositions embodying features of the present invention may be used, based on their NO attenuation or scavenging properties, as compounds for the treatment of diseases such as septic shock, rheumatoid arthritis, diabetes, asthma, and cancer.

Nitric oxide is an excellent ligand, especially with iron and ruthenium. Iron complexes of dithiocarbamates have been used as NO scavengers (Fujii, S. et al. Chem. Lett. 1996, 785). More recently, Cu(II) complexes of dithiocarbamates have also been studied as NO scavengers (Diaz, A. et al. J. Inorg. Biochem. 2003, 95, 283). It was found that the complex Cu(ProDTC)₂ (ProDTC=L-prolinedithiocarbamate) has an extremely high binding affinity for NO: log β₁=9.74 and log β₂=15.44. Ruthenium(III) complexes are of particular interest as NO scavengers, and have been investigated as immunosuppressive agents (Bastos, C. M. et al. Bioorg. Med. Chem. Lett. 1998, 8, 147), antitumor and anti-metastatic agents (Sava, G. et al. Top. Biol. Inorg. Chem. 1999, 1, 143; Sava, G. et al. Chem.-Biol. Interact. 1995, 95, 109; Sava, G. et al. Top. Biol. Met.-Based Drugs 1995, 2, 221), and nitric oxide (NO) scavengers (Cameron, B. R. Inorg. Chem. 2003, 42, 4102; Chatterjiee, D. et al. Dalton Trans. 2003, 203). Another aspect of the present invention provides the use of compounds embodying features of the present invention as sequestering agents for the treatment of heavy metal (e.g., Fe³⁺ and Pb²⁺) intoxication.

Methods of Making Crowned Dithiocarbamate Metal Complexes

Representative methods of making crowned dithiocarbamate metal complexes embodying features of the present invention are described in the Examples below. Variations of these methods may also be employed. Additional details for making compositions embodying features of the present invention are provided below.

Dithiocarbamates are sulfur-containing small molecules with extremely useful redox capabilities. DTCs have been used as heavy metal chelators. Synthesis of crowned DTC chelators was achieved by reacting the amino crown ether with carbon disulfide in the presence of a base, such as sodium hydroxide (Wang, J. H. and Wang, Y. L. Yingyong Huaxue 2002, 19, 295-297; and Wang, J.-H. and Zhang, Z. Yingyong Huaxue 1994, 11, 101). Since these crowned DTCs are slightly air-sensitive, they should be dried and stored under nitrogen. The size and the number of ether-oxygen donors can be systematically varied using synthetic techniques well known in the art.

DTC chelators form metal complexes with a variety of transition metal ions, including Fe²⁺, Fe³⁺, Co²⁺, Co³⁺, Cu²⁺, Mn²⁺, Zn²⁺, and Ru³⁺. It has been well-documented that DTC chelators form neutral ^{99m}Tc-nitrido complexes. Synthesis of neutral ^{99m}Tc-nitrido complexes of crowned DTCs may be achieved by following the literature methods (e.g., Marchi, A. et al. J. Chem. Soc. Dalton Trans. 1990, 1743; Duatti, A. et al. J. Chem. Soc., Dalton Trans. 1990, 3729; Pasqualini, R. et al. Appl. Radiat. Isot. 1992, 43, 1329; Pasqualini, R. et al. J. Nucl. Med. 1994, 35, 334; Zhang, J. et al. Nucl. Med. Biol. 2002, 29, 665; Zhang, J. et al. Appl. Radiat. Isot. 2002, 56, 857; Zhang, J. et al. Appl. Radiat. Isot. 2001, 54, 745; Zhang, J. and Wang, X. Appl. Radiat. Isot. 2001, 55, 453).

Macrocyclic crown ether containing groups have been the subject of intensive research for their ability to bind metal ions such as K⁺ and Na⁺ (Valeur, B. and Leray, I. Coord. Chem. Rev. 2000, 205, 3; Gunnlaugsson, T. and Leonard, J. P. J. Chem. Soc., Perkin Trans. 2002, 2, 1980). The extracellular Na⁺ concentration is 133-145 mM as compared to 3.5-4.8 mM for K⁺. However, the cytosolic Na⁺ concentration is only 10-40 mM as compared to 120 mM (upper limit) for K⁺ (Gunnlaugsson, T. and Leonard, J. P. J. Chem. Soc., Perkin Trans. 2002, 2, 1980; Gunnlaugsson, T. et al. J. Chem. Soc., Perkin Trans. 2002, 1, 1954). Although the 12- and 15-membered crown ether may not be able to form stable K⁺ complexes, the 18-membered crown ether group may result in a stronger interaction with K⁺. Therefore, the K⁺ binding capability may serve as a driving force for accumulation and retention of ^{99m}Tc complexes in myocardium. The selectivity for K⁺ may be tuned by changing the size and number of oxygen donor atoms of the macrocycle.

Duatti and coworkers (Bolzati, C. et al. J. Am. Chem. Soc. 2002, 124, 11468) have elegantly demonstrated that the heteroatom in the tridentate PXP (X=O and NR) ligand is required for stable asymmetrical ^{99m}Tc-nitrido complexes with the heteroatom invariably trans to the Tc≡N triple bond. The observed metal-heteroatom distances are quite long; this weak interaction seems to play a significant role in providing further stabilization for the ^{99m}Tc-nitrido core. These findings support the idea of using other tridentate chelators as tridentate co-ligands for the preparation of the mixed-ligand cationic ^{99m}Tc-nitrido complexes. The tripodal chelators of particular interest include but are not limited to the following examples:

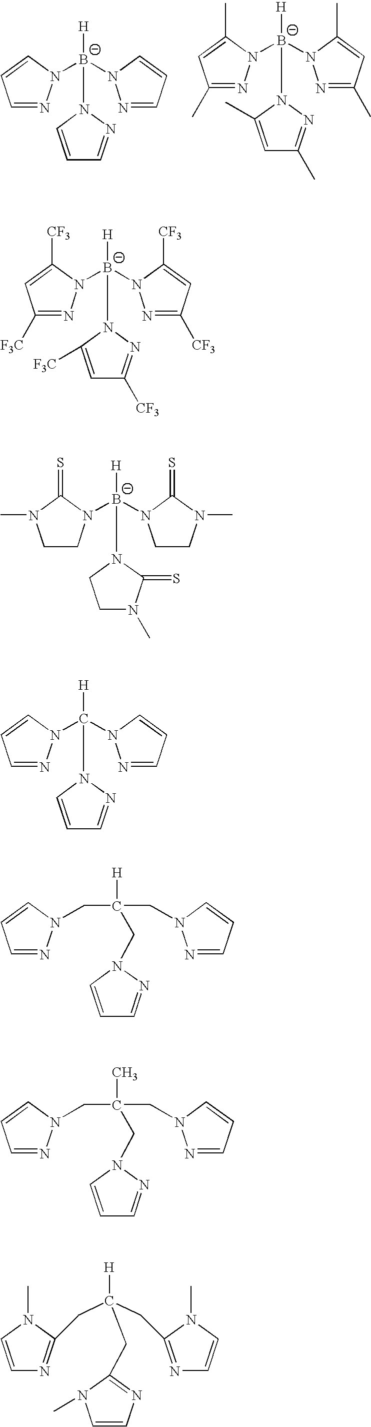

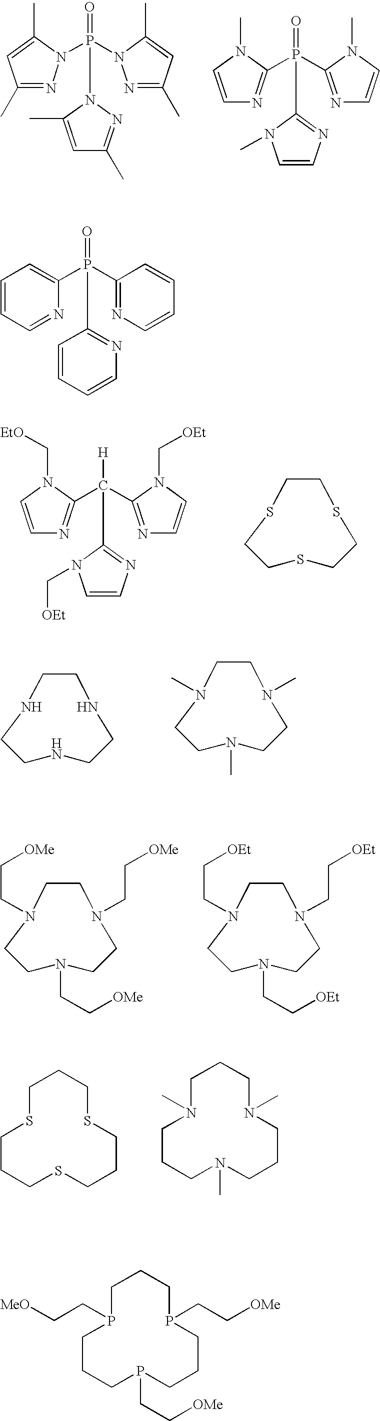

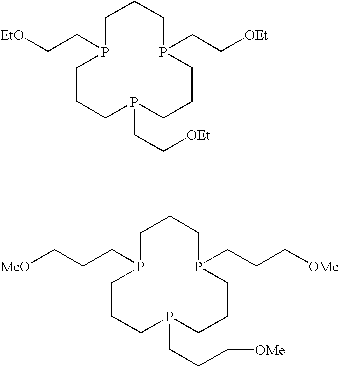

Like phosphine phosphorous, thioether sulfurs are π-acceptors. Although the Tc—S interaction is not as strong as the Tc—P (phosphine) bond, the macrocyclic effect will definitely enhance the stability of the fac-[^{99m}TcN([9]aneS₃)]²⁺ fragment. Imine N-containing heterocycles are π-acceptors. From this point of view, tridentate imine N containing tripodal chelators should also be used to form highly stable fac-[^{99m}TcN(N₃)]^{+/2+} fragments. The same basic idea should also apply to the fragment fac-[^{99m}TcN([9]aneN₃)]²⁺ except that [9]aneN₃ is not a π-acceptor. The exact structure of a particular radiometal complex in accordance with the present invention will depend on the identity of the tripodal coligand. In most cases, the tripodal coligand will occupy three coordination sites, and only one crowned DTC chelator will be needed to complete the octahedral coordination sphere of the radionuclide, such as ^{99m}Tc.

Zhang and coworkers (J. Labeled Compounds and Radiopharmaceuticals 2002, 45, 1029) reported the synthesis of a ^{99m}Tc-nitrido complex [^{99m}TcNCl₂(MIBI)₃]. Radio-HPLC data revealed only one peak for the complex [^{99m}TcNCl₂(MIBI)₃]. Although the exact composition for [^{99m}TcNCl₂(MIBI)₃] is not known, electrophoresis data suggested that it is most likely neutral with three MIBI ligands and two chloride anions bonding to the Tc. It was also found that one of the two chloride ligands is very labile, which was attributed to the partial cationic characteristics for the complex [^{99m}TcNCl₂(MIBI)₃] in solution.

In order to prevent partial dissociation, a bidentate dithiocarbamate chelator may be used to replace the two chlorides and form cationic ^{99m}Tc-nitrido complexes [^{99m}TcN(Isonitrile)₃(L)]⁺. The advantage of this new chelating system (three isonitriles and a dithiocarbamate) is the versatility of both isonitrile and dithiocarbamate ligands, which may be used for modification of physical and biological properties of their cationic ^{99m}Tc-nitrido complexes [^{99m}TcN(Isonitrile)₃(L)]⁺. If one crown ether group in complexes [^{99m}TcN(isonitrile)₃(L)]⁺ does not provide sufficient improvement in hydrophilicity, the combination of three crown ether-containing isonitriles with one crowned DTC will result in a significant improvement in lipophilicity of their cationic ^{99m}Tc-nitrido complexes.

The technetium and rhenium radionuclides are preferably in the chemical forms of [^{99m}Tc]pertechnetate or [^{186/188}Re]perrhenate and a pharmaceutically acceptable cation. The [^{99m}Tc]pertechnetate salt form is preferably sodium [^{99m}Tc]pertechnetate, such as may be obtained from commercial ^{99m}Tc generators. The amount of [^{99m}Tc]pertechnetate used to prepare metal complexes embodying features of the present invention may range from 1 mCi to 1000 mCi, or more preferably from 1 mCi to 50 mCi. Since there is no effective chemistry that can be used to attach the [^{99m}Tc]pertechnetate anion to an organic chelator, the [^{99m}Tc]pertechnetate is reduced by a reducing agent to a lower oxidation state in order to produce a stable ^{99m}Tc complex or to a reactive intermediate complex from which ^{99m}Tc can be easily transferred to the new chelator to form the expected ^{99m}Tc complex. Rhenium chemistry is very similar to technetium chemistry due to their periodic relationship. Therefore, the methods used for molecules labeled with ^{99m}Tc should apply to those labeled with ^{186/188}Re.

Suitable reducing agents for the synthesis of radiopharmaceuticals in accordance with the present invention include stannous salts, dithionite or bisulfite salts, borohydride salts, and formamidinesulfinic acid, wherein the salts are of any pharmaceutically acceptable form. A presently preferred reducing agent is a stannous salt. The amount of a reducing agent used may range from 0.001 mg to 10 mg, or more preferably from 0.005 mg to 1 mg.

The total time of preparation will vary depending on the metallic radionuclide, the identities and amounts of the reactants, and the procedure used for the preparation. The preparations may be complete and result in greater than 80% yield of metal complex in 1 minute or may require more time. After radiolabeling, the resulting reaction mixture may optionally be purified using one or more chromatographic methods, such as Sep-Pack or high performance liquid chromatography (HPLC). The preferred methods are those in which the ^{99m}Tc complex is prepared in high yield and high radiochemical purity without post-labeling purification.

The amounts of DTC chelator used for the preparation of radiometal chelates may range, for example, from 1 mg to 1000 mg, or more preferably from 1 mg to 10 mg. The exact amount of the DTC chelator is a function of the identity of a specific metal chelate, the procedure used for preparation of the metal chelate, and the amount and identities of the reactants used for the radiolabeling.

The compounds described herein may have one or more asymmetric centers. Compounds embodying features of the present invention containing an asymmetrically substituted atom may be isolated in optically active or racemic forms. It is well known in the art how to prepare optically active forms, such as by the resolution of racemic forms or by synthesis from optically active starting materials. All chiral, diastereomeric, racemic forms and all geometric isomeric forms of a structure are contemplated for use, unless the specific stereochemistry or isomeric form is specifically indicated. All processes used to prepare compounds of the present invention and intermediates made therein are considered to be part of the present invention.

Pharmaceutical Compositions and Kits Thereof

Kits and therapeutic compositions embodying features of the present invention, as well as various routes of therapeutically administering compositions embodying features of the present invention, will now be described.

In some embodiments, the present invention provides diagnostic kits for the preparation of radiopharmaceuticals useful as imaging agents for the diagnosis of cardiovascular disorders, infectious diseases, inflammatory diseases, and cancer. Diagnostic kits in accordance with the present invention may include one or more vials containing the sterile, non-pyrogenic, formulation comprised of a predetermined amount of a chelator in accordance with the present invention, a stabilizing coligand if needed, a reducing agent, and optionally other components, such as buffers, lyophilization aids, stabilization aids, solubilization aids and bacteriostats.

A radiopharmaceutical composition may contain, for example, the metal complex radiopharmaceutical, a buffer, a stabilization aid to prevent autoradiolysis, and a bacteriostat. If a radiopharmaceutical is prepared using the kit formulation, the radiopharmaceutical composition may contain the metal complex radiopharmaceutical and kit components, including unlabeled chelator, excess stabilizing coligand, a reducing agent, buffer, lyophilization aid, stabilization aid, solubilizing aids, and bacteriostats.

Buffers useful in the preparation of radiopharmaceuticals and in diagnostic kits useful for the preparation of the radiopharmaceuticals include but are not limited to phosphate, citrate, sulfosalicylate, and acetate. A more complete list may be found in the United States Pharmacopeia. Lyophilization aids useful in the preparation of diagnostic kits useful for the preparation of radiopharmaceuticals include but are not limited to mannitol, lactose, sorbitol, dextran, Ficoll, and polyvinylpyrrolidine (PVP).

Stabilization aids useful in the preparation of radiopharmaceuticals and in diagnostic kits useful for the preparation of the radiopharmaceuticals include but are not limited to ascorbic acid, cysteine, monothioglycerol, sodium bisulfite, sodium metabisulfite, gentisic acid, ascorbic acid, and inositol.

Solubilization aids useful in the preparation of radiopharmaceuticals and in diagnostic kits useful for the preparation of the radiopharmaceuticals include but are not limited to ethanol, glycerin, polyethylene glycol, propylene glycol, polyoxyethylene sorbitan monooleate, sorbitan monoloeate, polysorbates, poly(oxyethylene)poly(oxypropylene), poly(oxyethylene) block copolymers (Pluronics) and lecithin. Presently preferred solubilizing aids are polyethylene glycol and Pluronics.

Bacteriostats useful in the preparation of radiopharmaceuticals and in diagnostic kits useful for the preparation of the radiopharmaceuticals include but are not limited to benzyl alcohol, benzalkonium chloride, chlorbutanol, and methyl, propyl or butyl paraben.

A component in a diagnostic kit may also serve more than one function. By way of example, a reducing agent may also serve as a stabilization aid, a buffer may also serve as a transfer ligand, a lyophilization aid may also serve as a transfer, ancillary or coligand, and so forth.

The predetermined amounts of each component in the formulation are determined by a variety of considerations that are in some cases specific for that component and in other cases dependent on the amount of another component or the presence and amount of an optional component. In general, the minimal amount of each component is used that will give the desired effect of the formulation. The desired effect of the formulation is that the practicing end user may synthesize the radiopharmaceutical and have a high degree of certainty that the radiopharmaceutical may be safely injected into a patient and will provide diagnostic information about the disease state of that patient.

As used herein, the terms “kit” and “reagent kit” refer to an assembly of materials that are used in performing a method embodying features of the present invention. The reagents may be provided in packaged combination in the same or in separate containers, depending on their cross-reactivities and stabilities, and in liquid or in lyophilized form. The amounts and proportions of reagents provided in the kit may be selected so as to provide optimum results for a particular application.

Reagents included in kits embodying features of the present invention may be supplied in all manner of containers such that the activities of the different components are substantially preserved, while the components themselves are not substantially adsorbed or altered by the materials of the container. Suitable containers include but are not limited to ampoules, bottles, test tubes, vials, flasks, syringes, bags and envelopes (e.g., foil-lined), and the like. The containers may be comprised of any suitable material including but not limited to glass, organic polymers (e.g., polycarbonate, polystyrene, polyethylene, etc.), ceramic, metal (e.g., aluminum), metal alloys (e.g., steel), cork, and the like. In addition, the containers may contain one or more sterile access ports (e.g., for access via a needle), such as may be provided by a septum. Preferred materials for septa include rubber and polymers including but not limited to, for example, polytetrafluoroethylene of the type sold under the trade name TEFLON by DuPont (Wilmington, Del.). In addition, the containers may contain two or more compartments separated by partitions or membranes that can be removed to allow mixing of the components.

Kits embodying features of the present invention may also be supplied with other items known in the art and/or which may be desirable from a commercial and user standpoint, such as instructions for treating a tissue, a container for the tissue, diluents, preservation agents, antibiotics, antifungal drugs, antiviral drugs, anti-inflammatory drugs, surfactants, buffers, empty syringes, tubing, gauze, pads, disinfectant solution, etc. The diagnostic kits of the present invention may also contain instructions for the practicing end user to follow to synthesize radiopharmaceuticals embodying features of the present invention. In some embodiments, the instructions may be affixed to one or more of the containers (e.g., vials) or to a larger container in which one or more smaller containers are packaged for shipping. The instructions may also be provided as a separate insert, termed the package insert. The instructional materials may be printed (e.g., on paper) and/or supplied in an electronic-readable medium (e.g., floppy disc, CD-ROM, DVD-ROM, zip disc, videotape, audio tape, etc.). Alternatively, instructions may be provided by directing a user to an Internet web site (e.g., specified by the manufacturer or distributor of the kit) and/or via electronic mail.

In some embodiments, pharmaceutical compositions embodying features of the present invention comprise pharmaceutical carriers including but not limited to any sterile biocompatible pharmaceutical carrier such as saline, buffered saline, dextrose, water, and the like. Accordingly, in some embodiments, methods embodying features of the present invention comprise administering to a subject a pharmaceutical composition embodying features of the present invention in a suitable pharmaceutical carrier. In some embodiments, particular pharmaceutical compositions or therapies comprise a mixture of two or more different species of pharmaceutical compositions.

In some embodiments, the pharmaceutical compositions comprise a plurality of compositions administered to a subject under one or more of the following conditions: at different periodicities, different durations, different concentrations, or by different administration routes or the like.

In some embodiments, the pharmaceutical compositions and methods embodying features of the present invention may find use in treating diseases or altered physiological states characterized by pathogenic infection. However, the present invention is not limited to ameliorating (e.g., treating) any particular disease or infection. Indeed, various embodiments of the present invention are provided for treating (including prophylaxis) a range of physiological symptoms and disease etiologies in subjects including but limited to those characterized by aberrant cellular growth or proliferation (e.g., cancer), autoimmunity (e.g., rheumatoid arthritis), and other aberrant biochemical, genetic, and physiological symptoms. Depending on the condition being treated, the pharmaceutical compositions may be formulated and administered systemically or locally. Techniques for pharmaceutical formulation and administration are generally found in the latest edition of Remington's Pharmaceutical Sciences (Mack Publishing Co, Easton Pa.). Accordingly, the present invention contemplates administration of the pharmaceutical compositions in accordance with acceptable pharmaceutical delivery methods and preparation techniques.

In some embodiments, pharmaceutical compositions are administered to a subject (e.g., a patient) alone or in combination with one or more other drugs or therapies (e.g., antibiotics and antiviral agents, etc.) or in compositions where they are mixed with excipients or other pharmaceutically acceptable carriers.

Generally, pharmaceutical compositions in accordance with the present invention may be delivered via any suitable method, including but not limited to orally, intravenously, subcutaneously, intratumorally, intraperitoneally, or topically (e.g., to mucosal surfaces).

In some embodiments, pharmaceutical compositions embodying features of the present invention are formulated for parenteral administration, including intravenous, subcutaneous, intramuscular, and intraperitoneal administration. Some of these embodiments comprise a pharmaceutically acceptable carrier, such as physiological saline. For injection, the pharmaceutical compositions are typically formulated in aqueous solution, preferably in physiologically compatible buffers (e.g., Hanks' solution, Ringer's solution, or physiologically buffered saline). For tissue or cellular administration, penetrants appropriate to the particular barrier to be permeated are also preferable. Such penetrants are well known in the art. Other embodiments use standard intracellular delivery (e.g., delivery via liposomes) techniques. Intracellular delivery methods are well known in the art. Administration of some agents to a patient's bone marrow may necessitate delivery in a manner different from intravenous injections.

In some embodiments, active pharmaceutical compositions are prepared as oily injection suspensions. Suitable lipophilic solvents or vehicles include but are not limited to fatty oils such as sesame oil, synthetic fatty acid esters, such as ethyl oleate or triglycerides, or liposomes. Aqueous injectable suspensions may additionally comprise substances that increase the viscosity of the suspension, such as sodium carboxymethyl cellulose, sorbitol, and dextran. Optionally, the injectable suspension may also comprise suitable stabilizers and agents that increase or prolong the solubility of the compounds, thus allowing preparation of highly concentrated solutions.

In some embodiments, pharmaceutical compositions in accordance with the present invention are formulated using pharmaceutically acceptable carriers in suitable dosages for oral administration. Suitable carriers enable the compositions to be formulated as tablets, pills, capsules, dragees, liquids, gels, syrups, slurries, suspensions and the like, for oral or nasal ingestion by a subject.

In some embodiments, pharmaceutical compositions for oral use are made by combining the active compounds (e.g., chemical address tag-therapeutic agent conjugates) with a solid excipient, optionally grinding the resulting mixture, and processing the mixture of granules, after adding suitable auxiliaries, if desired, so as to obtain tablets or dragee cores. Suitable excipients include but are not limited: carbohydrate fillers such as sugars, including, lactose, sucrose, mannitol, or sorbitol; starch from corn, wheat, rice, potato; cellulose such as methyl cellulose, hydroxypropylmethylcellulose, and sodium carboxymethylcellulose; gums including arabic and tragacanth; and proteins such as gelatin and collagen. If desired, disintegrating or solubilizing agents may be added, such as cross-linked polyvinyl pyrrolidone, agar, alginic acid or a salt thereof, such as sodium alginate.

Ingestible formulations of pharmaceutical compositions in accordance with the present invention may further comprise any material approved by the United States Department of Agriculture (or other similar international agency) for inclusion in foodstuffs and substances that are generally recognized as safe (GRAS), such as food additives, flavorings, colorings, vitamins, minerals, and phytonutrients. As used herein, the term refers to organic compounds isolated from plants that have a biological effect, including but not limited to compounds in the following classes: isoflavonoids, oligomeric proanthcyanidins, indol-3-carbinol, sulforaphone, fibrous ligands, plant phytosterols, ferulic acid, anthocyanocides, triterpenes, omega 3/6 fatty acids, polyacetylene, quinones, terpenes, cathechins, gallates, quercitin, and the like.

Preferably, dragee cores are provided with suitable coatings such as concentrated sugar solutions, which may contain gum arabic, talc, polyvinylpyrrolidone, carbopol gel, polyethylene glycol, titanium dioxide, lacquer solutions, and suitable organic solvents or solvent mixtures. Dyestuffs or pigments may be added to tablets or dragee coatings for product identification or to characterize the quantity of active compound, (i.e., dosage).

Orally formulated compositions embodying features of the present invention may include but are not limited to push-fit capsules (e.g., those made of gelatin) and soft sealed capsules (e.g., those made of gelatin), optionally having a coating such as glycerol or sorbitol. Push-fit capsules may contain active ingredients mixed with fillers or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol, with or without stabilizers. In presently preferred embodiments, the pharmaceutically acceptable carriers are pharmaceutically inert.

In some embodiments, pharmaceutical compositions used in methods embodying features of the present invention are manufactured according to well-known and standard pharmaceutical manufacturing techniques (e.g., by means of conventional mixing, dissolving, granulating, dragee-making, levigating, emulsifying, encapsulating, entrapping or lyophilizing processes).

Pharmaceutical compositions suitable for use in accordance with the present invention may further include compositions wherein the active ingredient(s) is/are contained in an effective amount to achieve the intended purpose. A therapeutically effective dose refers to that amount of a composition(s) that reduces symptoms or eliminates at least one symptom of the disease state. For example, an effective amount of a therapeutic compound(s) may be that amount that destroys or disables pathogens as compared to a control.

Dosing is dependent on severity and responsiveness of the disease state to be treated, with the course of treatment lasting from several days to several months, or until a cure is effected or a diminution of the disease state is achieved. Guidance as to particular dosing considerations and methods of delivery are provided in the literature (See, e.g., U.S. Pat. Nos. 4,657,760; 5,206,344; and 5,225,212). Optimal dosing schedules are calculated from measurements of composition accumulation in the subject's body. The administering physician may easily determine optimum dosages, dosing methodologies and repetition rates. Optimum dosages may vary depending on the relative potency of compositions and may generally be estimated based on the EC₅₀ values found to be effective in in vitro and in vivo animal models. Additional factors that may be taken into account include but are not limited to the severity of the disease state; the subject's age, weight, and gender; the subject's diet; the time and frequency of administration; combination(s) of agents or compositions; possible reaction sensitivities or allergies; and the subject's tolerance/response to prior treatments. In general, dosage is from 0.001 μg to 100 g per kg of body weight, and may be given once or more daily, weekly, monthly or yearly. The treating physician preferably estimates dosing repetition rates based on measured residence times and concentrations of the agents/drugs in the subject's fluids or tissues. Following successful treatment, it may be desirable to have the subject undergo maintenance therapy to prevent the recurrence of the disease state, wherein the therapeutic agent is administered in maintenance doses, ranging from 0.001 μg to 100 g per kg of body weight, once or more daily, weekly, or the like.

For any pharmaceutical composition used in methods embodying features of the present invention, the therapeutically effective dose may be estimated initially from cell culture assays. Then, preferably, dosages may be formulated in animal models (e.g., murine or rat models) to achieve a desirable circulating concentration range.

Toxicity and therapeutic efficacy of administered pharmaceutical compositions may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which may be expressed as the ratio LD₅₀/ED₅₀. Compounds that exhibit large therapeutic indices are preferred. The data obtained from cell culture assays and additional animal studies may be used in formulating a range of dosages, for example, for mammalian use (e.g., in humans). The dosage of such compounds preferably lies within a range of circulating concentrations that include the ED₅₀ with little or no toxicity. However, the present invention is not limited to this range.

### EXPERIMENTAL

The following examples are provided in order to demonstrate and further illustrate certain preferred embodiments and aspects of the present invention and are not to be construed as limiting the scope thereof. In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); DS (dextran sulfate); and C (degrees Centigrade).

Instruments

Chemicals were purchased from Sigma/Aldrich (St. Louis). ¹H NMR spectra were recorded on a 300 MHz Bruker spectrometer. The ¹H NMR data are reported as δ (ppm) relative to TMS.

### Example I—Synthesis of Sodium Dithiocarbamate of 2-Aminomethyl-15-Crown-5

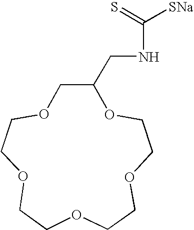

To a cold solution of 2-aminomethyl-15-crown-5 (0.524 g, 2.0 mmol) and sodium hydroxide (0.08 g, 2.0 mmol) in 6 mL of ethanol was added carbon disulfide (0.304 g, 2.0 mmol) dropwise. The resulting mixture was stirred at 0-5° C. for another 2-3 h, during which time a white precipitate was formed. Upon addition of 40 mL of diethyl ether and vigorous stirring, more precipitate was formed. The mixture was kept still and the supernatant was decanted. The white precipitate was washed twice with ether (2×10 mL) and dried under vacuum overnight to give the product as a white solid in quantitative yield. ¹H NMR (D₂O, chemical shift δ in ppm): 3.80-3.72(m, 2H), 3.64-3.45 (m, 17H), and 3.41 (m, 2H). ¹³C NMR (D₂O, DMSO-d₆ as internal reference, chemical shift δ in ppm): 214.0 (C═S), 78.1, 72.0, 71.1, 71.0, 70.8, 70.7, 70.6, 70.5, 70.4, 70.3 (crown ether carbons), and 49.4 (CH₂N).

### Example II—Synthesis of Sodium Dithiocarbamate of 2-Aminomethyl-18-Crown-6

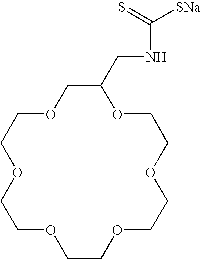

A solution of 2-aminomethyl-18-crown-6 (0.293 g, 1.0 mmol) in 1 mL of ethanol was dropwise added into a cold solution of carbon disulfide (0.076 g, 1.0 mmol) and sodium hydroxide (0.04 g, 1.0 mmol) in 2 mL of ethanol with stirring. The resulting mixture was stirred at 0-5° C. for another 2-3 h. Then 30 mL of ether was gradually added with vigorous stirring and the desired dithiocarbamate product was separated as a pale yellow oil, which was further washed twice with ether (2×10 mL) and dried under vacuum to give the product as a foamy solid (0.37 g, yield 95%). ¹H NMR (D₂O, chemical shift δ in ppm): 3.78 (m, 2H), 3.66-3.40 (m, 21H), and 3.37 (m, 2H). ¹³C NMR (D₂O, DMSO-d₆ as internal reference, chemical shift δ in ppm): δ 197.0 (C═S), 78.5, 72.0, 71.5, 71.0, 70.9, 70.8 (m), 70.2, 70.0 (crown ether carbons), and 47.7 (CH₂N).

### Example III—Synthesis of Sodium Dithiocarbamate of 3-Aminobenzo-15-Crown-5

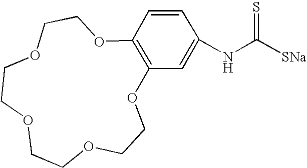

To a cold solution of 4′-aminobenzo-15-crown-5 (0.283 g, 1.0 mmol) and sodium hydroxide (0.04 g, 1.0 mmol) in 5 mL of ethanol was added carbon disulfide (0.076 g, 1.0 mmol) dropwise. The resulting mixture was stirred at 0-5° C. for another 2-3 h. Upon addition of 20 mL of diethyl ether with vigorous stirring, a grey precipitate was formed, which was isolated by filtration, washed twice with ether (2×10 mL) and dried under vacuum to give the product as a grey solid (0.28 g, yield 74%). ¹H NMR (D₂O, chemical shift δ in ppm): 6.73 (d, 1H, J=8.5 Hz), 6.38 (d, 1H, J=2.5 Hz), 6.25 (dd, 1H, J=8.5 and 2.5 Hz), 3.95 (m, 4H), 3.70 (m, 4H), and 3.60-3.55 (m, 8H).

### Example IV—Synthesis of Sodium Dithiocarbamate of 4′-Amino-5′-Nitrobenzo-15-crown-5

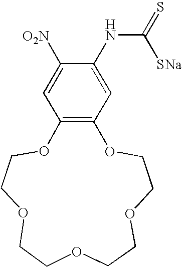

To a cold solution of 4′-amino-5′-nitrobenzo-15-crown-5 (0.328 g, 1.0 mmol) and sodium hydroxide (0.04 g, 1.0 mmol) in 5 mL of ethanol was added carbon disulfide (0.076 g, 1.0 mmol) dropwise. Upon addition of the carbon disulfide, a yellow precipitate was formed within 10 min. The resulting mixture was stirred at 0-5° C. for another 2-3 h. Upon addition of 20 mL of diethyl ether with vigorous stirring, a golden yellow precipitate was formed, which was isolated by decanting the upper supernatant. Then it was washed twice with ether (2×10 mL) and dried under vacuum to give the product as a golden yellow solid (0.41 g, yield 96%). ¹H NMR (D₂O, chemical shift δ in ppm): 7.33 (s, 1H), 6.25 (s, 1H), 4.07-3.95 (m, 4H), 3.75 (m, 4H), and 3.60-3.53 (m, 8H).

### Example V—Synthesis of Sodium Dithiocarbamate of 1-Aza-15-Crown-5

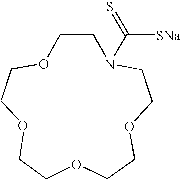

To a cold solution of 2-aminomethyl-15-crown-5 (0.524 g, 2.0 mmol) and sodium hydroxide (0.08 g, 2.0 mmol) in 6 mL of ethanol was added carbon disulfide (0.304 g, 2.0 mmol) dropwise. The resulting mixture was stirred at 0-5° C. for another 2-3 h, during which time a white precipitate was formed. Upon addition of 40 mL of diethyl ether with vigorous stirring, more precipitate was formed. The mixture was kept still and the supernatant was decanted. The white precipitate was washed twice with ether (2×10 mL) and dried under vacuum overnight to give the product as a white solid in quantitative yield. ¹H NMR (D₂O, chemical shift δ in ppm): 4.20 (t, 4H, J=6.0 Hz), 3.70 (t, 4H, J=6.0 Hz), and 3.60 (m, 12H). ¹³C NMR (D₂O, DMSO-d₆ as internal reference, chemical shift δ in ppm): 206.1 (C═S), 71.2, 70.8, 69.5, and 56.8 (crown ether ring carbons).

### Example VI—Synthesis of Sodium Dithiocarbamate of 1-Aza-18-Crown-6

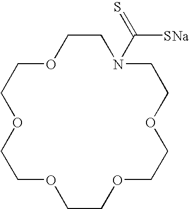

To a cold solution of 1-aza-18-crown-6 (0.276 g, 95%, 1.0 mmol) and sodium hydroxide (0.04 g, 1.0 mmol) in 3 mL of ethanol was added carbon disulfide (0.076 g, 1.0 mmol) dropwise. The resulting mixture was stirred at 0-5° C. for another 2-3 h. Upon addition of 20 mL of diethyl ether with vigorous stirring, a white precipitate was formed, which was isolated by decanting the upper supernatant. Then it was washed twice with ether (2×10 mL) and dried under vacuum to give the product as a white solid (0.32 g, yield 84%). ¹H NMR (D₂O, chemical shift δ in ppm): 4.19 (t, 4H, J=6.0 Hz), 3.70 (t, 4H, J=6.0 Hz), and 3.52 (m, 16H). ¹³C NMR (D₂O, DMSO-d₆ as internal reference, chemical shift δ in ppm): 205.7 (C═S), 71.9, 70.9 (m), 69.5, 56.7 (crown ether ring carbons).

### Example VII—Synthesis of ^{99m}Tc-Nitrido Complex of 1-(Aza-12-Crown-4)Dithiocarbamate

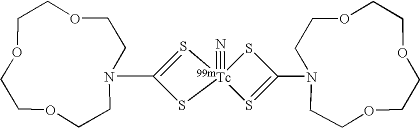

The solution containing succinic dihydrazide (SDH) and propylenediaminetetraacetic acid (PDTA) was prepared according to the literature procedure (Zhang, J. et al. J. Labelled Compounds & Radiopharm. 2000, 43, 693-700). To a 5-mL vial was added the solution containing PDTA (5 mg/mL) and SDH (5 mg/mL), followed by addition of 1.0 mL saline containing 2.0 mCi of ^{99m}Tc-pertechnetate, and 10 μL SnCl₂ solution in 1.0 N HCl. The reaction mixture was kept at room temperature for 15 min to give the ^{99m}Tc-nitrido intermediate. After addition of 0.5 mL of solution containing sodium 1-(aza-12-Crown-4)-dithiocarbamate (10 mg/mL), the reaction mixture was allowed to stand at room temperature for another 15 min. The radiochemical purity (RCP) was evaluated by radio-HPLC. The retention time was 7.5 min. The RCP was >95%.

### Example VIII—Synthesis of ^{99m}Tc-Nitrido Complex of 1-(Aza-15-Crown-5)Dithiocarbamate

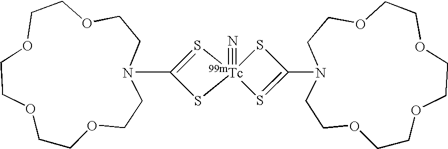

To a 5-mL vial was added the solution containing PDTA (5 mg/mL) and SDH (5 mg/mL), followed by addition of 1.0 mL saline containing 2.0 mCi of ^{99m}Tc-pertechnetate and 10 μL SnCl₂ solution in 1.0 N HCl. The reaction mixture was kept at room temperature for 15 min to give the ^{99m}Tc-nitrido intermediate. After addition of 0.5 mL of solution containing sodium 1-(aza-15-crown-5)-dithiocarbamate (10 mg/mL), the reaction mixture was allowed to stand at room temperature for another 15 min. The resulting solution was analyzed by radio-HPLC. The retention time was 7.3 min. The RCP was >95%.

### Example IX—Synthesis of ^{99m}Tc-Nitrido Complex of 1-(Aza-18-Crown-6)Dithiocarbamate

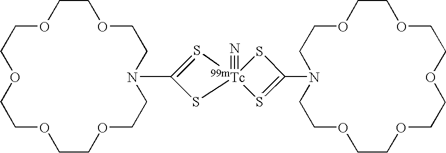

To a 5-mL vial was added the solution containing PDTA (5 mg/mL) and SDH (5 mg/mL), 1.0 mL saline containing 2.0 mCi of ^{99m}Tc-pertechnetate, and 10 μL SnCl₂ solution in 1.0 N HCl. The reaction mixture was kept at room temperature for 15 min. After addition of 0.5 mL of solution containing sodium 1-(aza-18-crown-6)-dithiocarbamate (10 mg/mL), the reaction mixture was kept at room temperature for 15 min. The resulting solution was analyzed by radio-HPLC. The retention time was 7.1 min. The RCP was >95%.

### Example X—Synthesis of ^{99m}Tc-Nitrido Complex of N-(1-Aminomethyl-15-Crown-5)Dithiocarbamate

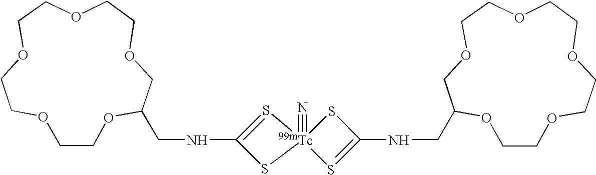

To a 5-mL vial was added the solution containing PDTA (5 mg/mL) and SDH (5 mg/mL), 1.0 mL saline containing 2.0 mCi of ^{99m}Tc-pertechnetate, and 10 μL SnCl₂ solution in 1.0 N HCl. The reaction mixture was kept at room temperature for 15 min. After addition of 0.5 mL of solution containing sodium N-(1-aminomethyl-15-Crown-5)-dithiocarbamate (10 mg/mL), the reaction mixture was kept at room temperature for 15 min. The resulting solution was analyzed by radio-HPLC. The retention time was 7.4 min. The RCP was >95%.

### Example XI—In Vivo Testing

The compounds of the present invention may be tested in vivo to determine the therapeutic characteristics of these compounds and to identify particular compounds with desired characteristics. For example, animal studies may be performed following the literature procedures (e.g., Boschi, A. et al. Nucl. Med. Commun. 2002, 23, 689). Biodistribution studies may be carried out using Sprague-Dawley rats in compliance with NIH animal experiment guidelines (Principles of Laboratory Animal Care, NIH Publication No. 86-23, revised 1985). One objective of these studies is to determine the characteristics of the ^{99m}Tc complexes (or other compounds embodying features of the present invention) as radiopharmaceuticals. These studies may be used as a preliminary screening tool to determine the biodistribution characteristics, excretion kinetics, and metabolism of ^{99m}Tc complexes of crowned DTC chelators.

Sprague-Dawley rats (200-250 g) may be anesthetized with an intramuscle injection of a mixture of ketamine (80 mg/kg) and xylazine (19 mg/kg). These rats may then receive the ^{99m}Tc radiopharmaceutical (50-100 μCi in 100 μL solution) via intravenous injection into the tail vein. The amount of activity administered to each animal may be quantified, ultimately allowing the biodistribution of each radiopharmaceutical to be calculated as both a percentage of the injected dose per organ (% ID/organ) and a percentage of the injected dose per gram of tissue wet mass (% ID/g). The animals may then be sacrificed via exsanguinations and opening of the thoracic cavity at selected time points. Relevant tissues and organs may then be excised, weighed, and counted to determine the tissue uptake of the ^{99m}Tc complex (or other compounds in accordance with the present invention that are tested). The organs that may be examined include, for example, heart, brain, blood, lung, liver, spleen, kidneys, muscle, intestines, and bone. Five rats, for example, may be used at each selected time point to ensure acquisition of reliable biological data. Statistical analysis of the biodistribution data from these experiments may employ the Student t test for comparison of radiotracer biodistribution between different ^{99m}Tc radiopharmaceuticals (or other compounds in accordance with the present invention). For comparison purposes, biodistribution studies may also be performed on both ^{99m}Tc-Sestamibi and ^{99m}Tc-Tetrofosmin, which are two radiopharmaceuticals approved for myocardial perfusion imaging. Preferred ^{99m}Tc radiopharmaceuticals would be those that have high heart uptake, long heart retention time, and rapid blood clearance, preferably via renal system.

This model may also be used to assess the effectiveness of radiopharmaceuticals embodying features of the present invention comprised of a positron emitting isotope such as ^{94m}Tc. The radiopharmaceuticals may be administered in appropriate amounts and the uptake in the heart may be quantified non-invasively by imaging for those isotopes with a coincident imageable gamma emission. The diagnostic radiopharmaceuticals may be administered by intravenous injection, usually in saline solution, at a dose of 1 to 100 mCi per 70 kg body weight, or preferably at a dose of 5 to 30 mCi. Imaging is performed using known procedures. The therapeutic radiopharmaceuticals may be administered by intravenous injection, usually in saline solution, at a dose of 0.1 to 100 mCi per 70 kg body weight, or preferably at a dose of 0.5 to 50 mCi per 70 kg body weight.

The foregoing detailed description and examples have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. A composition comprising a compound comprising a formula (M≡N)L¹ and pharmaceutically acceptable salts thereof;
wherein N is nitrogen;
M is a transition metal; and
L¹ is a first crowned dithiocarbamate, wherein the first crowned dithiocarbamate comprises a first crown ether-containing group of formula [(CH₂)ₐ—O]_{b}—(CH₂)_{c}, wherein a is at least 2, b is at least 3, and c is at least 2.

2. The composition of claim 1, wherein the first crowned dithiocarbamate comprises a formula: 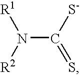
and pharmaceutically acceptable salt thereof;
wherein R¹ or R² comprises the first crown ether-containing group, or R¹ and R² together comprise the first crown ether-containing group.

3. The composition of claim 1, wherein the transition metal is a radioactive metal.

4. The composition of claim 1, wherein the transition metal is ^{99m}Tc or ^{94m}Tc.

5. The composition of claim 1, wherein the transition metal is ¹⁸⁶Re or ¹⁸⁸Re.

6. The composition of claim 1, wherein subscript a in the formula of the first crown ether-containing group is 2, 3, 4, or 5.

7. The composition of claim 1, wherein subscript a in the formula of the first crown ether-containing group is 2 or 3.

8. The composition of claim 1, wherein subscript a in the formula of the first crown ether-containing group is 2.

9. The composition of claim 1, wherein subscript b in the formula of the first crown ether-containing group is 3, 4, 5, 6, 7, or 8.

10. The composition of claim 1, wherein subscript b in the formula of the first crown ether-containing group is 3, 4, 5 or 6.

11. The composition of claim 1, wherein subscript c in the formula of the first crown ether-containing group is 2, 3, 4, or 5.

12. The composition of claim 1, wherein subscript c in the formula of the first crown ether-containing group is 2 or 3.

13. The composition of claim 1, wherein subscript c in the formula of the first crown ether-containing group is 2.

14. The composition of claim 1, wherein the first crowned dithiocarbamate comprises

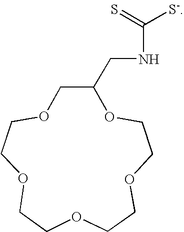

15. The composition of claim 1, wherein the first crowned dithiocarbamate comprises

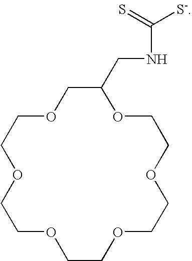

16. The composition of claim 1, wherein the first crowned dithiocarbamate comprises

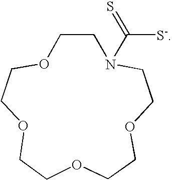

17. The composition of claim 1, wherein the first crowned dithiocarbamate comprises

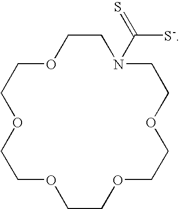

18. The composition of claim 1, wherein the first crowned dithiocarbamate comprises

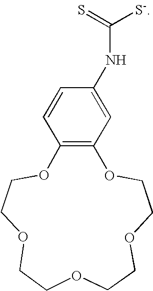

19. The composition of claim 1, wherein the first crowned dithiocarbamate comprises

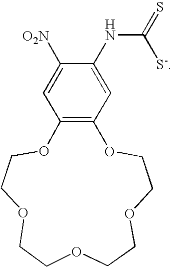

20. The composition of claim 1, wherein the first crowned dithiocarbamate is selected from the group consisting of

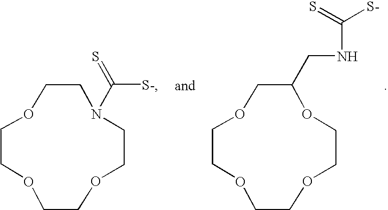

21. The composition of claim 1, wherein the compound is selected from the group consisting of:

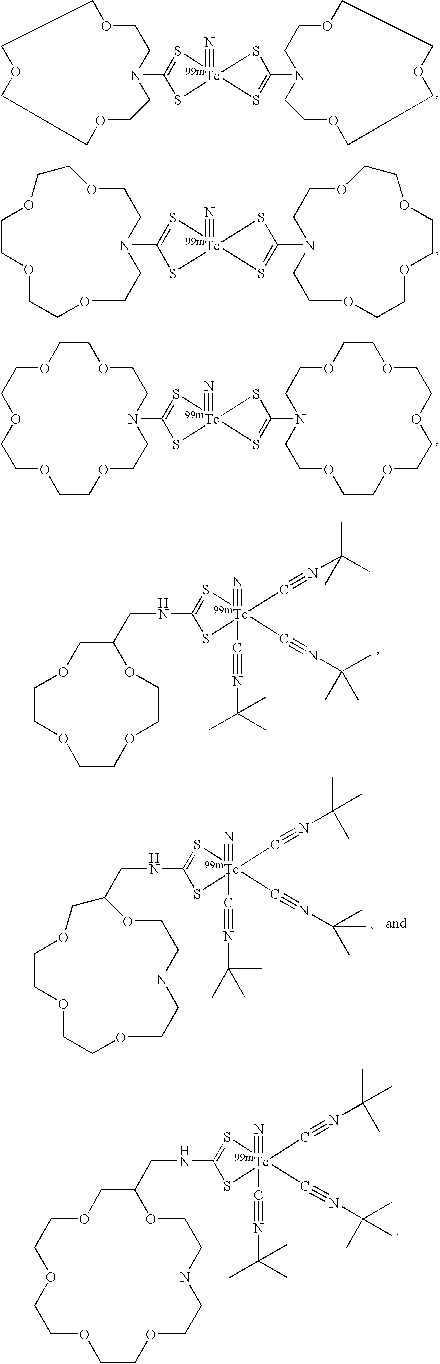

22. The composition of claim 1, wherein the compound further comprises L² and comprises a formula (M≡N)L¹L² and pharmaceutically acceptable salts thereof;
wherein L² is a second crowned dithiocarbamate, wherein the second crowned dithiocarbamate comprises a second crown ether-containing group of formula [(CH₂)ₐ—O]_{b}—(CH₂)_{c}, wherein a is at least 2, b is at least 3, and c is at least 2.

23. The composition of claim 22, wherein the second crowned dithiocarbamate comprises a formula: 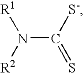
and pharmaceutically acceptable salt thereof;
wherein R¹ or R² comprise the second crown ether-containing group, or R¹ and R² together comprise the second crown ether-containing group.

24. The composition of claim 22, wherein subscript a in the formula of the second crown ether-containing group is 2, 3, 4, or 5.

25. The composition of claim 22, wherein subscript a in the formula of the second crown ether-containing group is 2 or 3.

26. The composition of claim 22, wherein subscript a in the formula of the second crown ether-containing group is 2.

27. The composition of claim 22, wherein subscript b in the formula of the second crown ether-containing group is 3, 4, 5, 6, 7, or 8.

28. The composition of claim 22, wherein subscript b in the formula of the second crown ether-containing group is 3, 4, 5 or 6.

29. The composition of claim 22, wherein subscript c in the formula of the second crown ether-containing group is 2, 3, 4, or 5.

30. The composition of claim 22, wherein subscript c in the formula of the second crown ether-containing group is 2 or 3.

31. The composition of claim 22, wherein subscript c in the formula of the second crown ether-containing group is 2.

32. The composition of claim 22, wherein the second crowned dithiocarbamate comprises

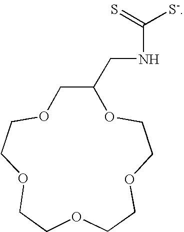

33. The composition of claim 22, wherein the second crowned dithiocarbamate comprises

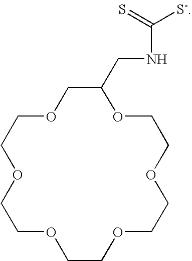

34. The composition of claim 22, wherein the second crowned dithiocarbamate comprises

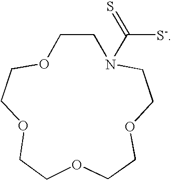

35. The composition of claim 22, wherein the second crowned dithiocarbamate comprises

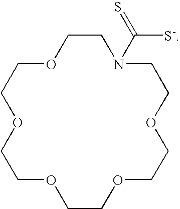

36. The composition of claim 22, wherein the second crowned dithiocarbamate comprises

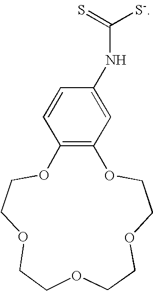

37. The composition of claim 22, wherein the second crowned dithiocarbamate comprises

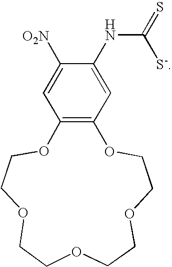

38. The composition of claim 22, wherein the second crowned dithiocarbamate is selected from the group consisting of

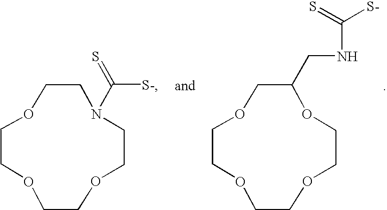

39. The composition of claim 1, wherein the compound is selected from the group consisting of:

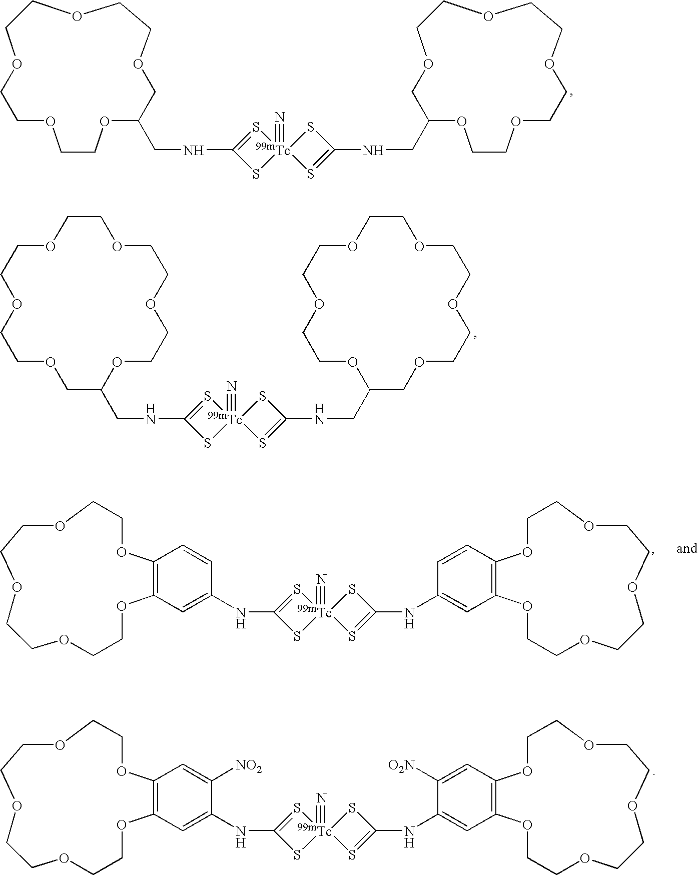

40. The composition of claim 1, wherein the compound further comprises L³, L⁴ and L⁵ and comprises a formula: 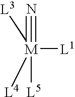
and pharmaceutically acceptable salts thereof;
wherein L³, L⁴, and L⁵ each comprises an isonitrile of formula: 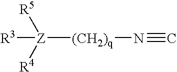
wherein q is 0-3;
Z is carbon or silicon;
R³, R⁴ and R⁵ are the same or different, and are selected from the group consisting of H, C₁-C₁₀ alkyl substituted with 0-5 R⁶, aryl substituted with 0-5 R⁶, heteroaryl substituted with 0-5 R⁶, and macrocyclic crown ether containing 2-8 ether-oxygen atoms;
wherein R⁶ is selected from the group consisting of H, OH, OR⁷, C(═O)OR⁷, C(═O)NR⁸R⁹, PO(OR⁸)₂, PO(NR⁸R⁹)₂ and SO₂R⁷; and
R⁷, R⁸ and R⁹ are the same or different, and are selected from the group consisting of H, alkyl, aryl, and heteroaryl, or R⁸ and R⁹ together form a macrocyclic crown ether containing 2-8 ether-oxygen atoms.

41. The composition of claim 1, wherein the compound further comprises L³, L⁴, and L⁵ and comprises a formula: 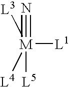
and pharmaceutically acceptable salts thereof;
wherein L³, L⁴, and L⁵ together form a tripodal chelator of formula: 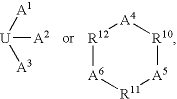
wherein U is selected from the group consisting of R¹³B, CR¹³ and P(═O);
A¹, A² and A³ are imine-N containing heterocycles;
A⁴, A⁵ and A⁶ are selected from the group consisting of NR¹⁴, PR¹⁴, S, and O;
R¹⁰, R¹¹ and R¹² are selected from a group of formula —(CH₂)_{g}—, wherein g is 2-5;
R¹³ is selected from the group consisting of H, alkyl and aryl; and
R¹⁴ is selected from the group consisting of H, alkyl, aryl, and alkoxyalkyl.

42. The composition of claim 41, wherein the tripodal chelator is selected from the group consisting of

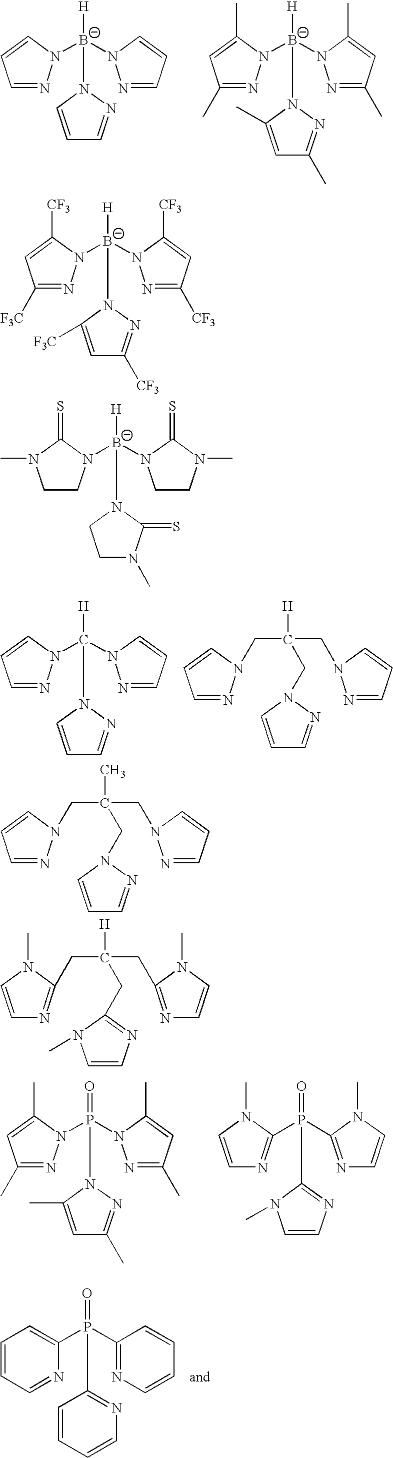

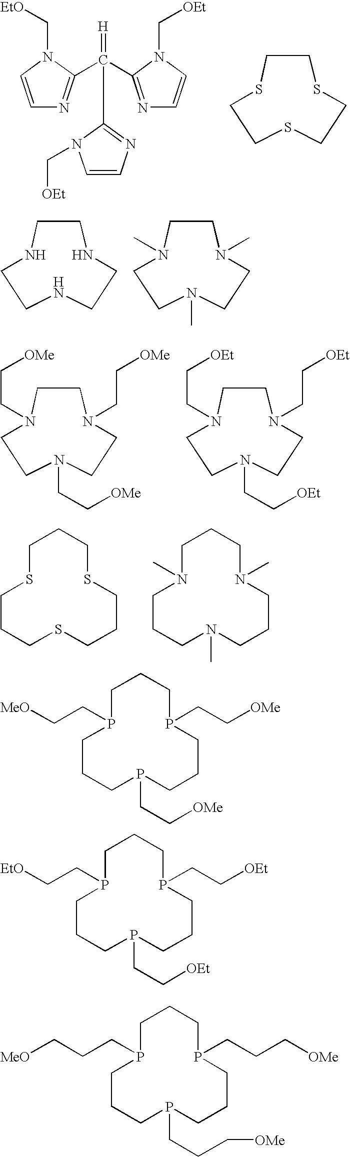

43. A composition comprising a compound comprising a formula: 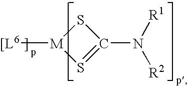
and pharmaceutically acceptable salt thereof;
wherein M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II);
p and p′ are integers and are independently selected from 0-2;
R¹ and R² comprise a crown ether-containing group of formula [(CH₂)ₐ—O]_{b}—(CH₂)_{c}, wherein a is at least 2, b is at least 3, and c is at least 2, or wherein R¹ and R² together comprise the crown ether-containing group;
L⁶ is a tripodal chelator with a formula selected from the group consisting of 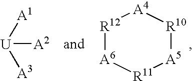
wherein U is selected from the group consisting of R¹³B, CR¹³, and P(═O);
A¹, A² and A³ are imine-N containing heterocycles;
A⁴, A⁵ and A⁶ are selected from the group consisting of NR¹⁰, PR¹⁰, and S;
R¹⁰, R¹¹ and R¹² are selected from a group of formula —(CH₂)_{g}—, wherein g is 2-5;
R¹³ is selected from the group consisting of H, alkyl and aryl; and
R¹⁴ is selected from the group consisting of H, alkyl, aryl, and alkoxyalkyl.

44. A method for radioimaging a subject comprising:
a) providing
i) a subject; and
ii) a composition comprising a compound comprising a formula (M≡N)L¹ and pharmaceutically acceptable salts thereof;
b) administering the composition to the subject; and
c) scanning at least a portion of the subject using a radioimaging device;
wherein N is nitrogen;
M is a radioactive transition metal; and
L¹ is a first crowned dithiocarbamate, wherein the first crowned dithiocarbamate comprises a first crown ether-containing group of formula [(CH₂)ₐ—O]_{b}—(CH₂)_{c}, wherein a is at least 2, b is at least 3, and c is at least 2.

45. The method of claim 44, wherein at least a portion of the subject is tissue suspected of being diseased.

46. The method of claim 44, wherein the at least a portion of the subject is myocardial tissue.

47. The method of claim 44, wherein the subject is a mammal.

48. A method of treating a disease resulting from overproduction of nitric oxide or reactive oxygen species, comprising:
a) providing:
i) a subject with a disease; and
ii) a composition comprising a compound comprising a formula: 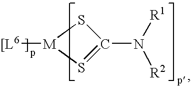
and pharmaceutically acceptable salt thereof; and
b) administering the composition to the subject;
wherein M is a transition metal selected from the group consisting of Fe(II), Fe(III), Mn(II), Mn(III), Co(II), Co(III), Ni(II), Cu(II), Zn(II), Ru(II), Ru(III), Pd(II), and Pt(II);
p and p′ are integers and are independently selected from 0-2;
R¹ and R² comprises a crown ether-containing group of formula [(CH₂)ₐ—O]_{b}—(CH₂)_{c}, wherein a is at least 2, b is at least 3, and c is at least 2, or wherein R¹ and R² together comprise the crown ether-containing group;
L⁶ is a tripodal chelator with a formula selected from the group consisting of: 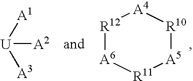
wherein U is selected from the group consisting of R¹³B, CR¹³, and P(═O);
A¹, A² and A³ are imine-N containing heterocycles;
A⁴, A⁵ and A⁶ are selected from the group consisting of NR¹⁰, PR¹⁰, and S;
R¹⁰, R¹¹ and R¹² are selected from a group of formula (CH₂)_{g}—, wherein g is 2-5;
R¹³ is selected from the group consisting of H, alkyl and aryl; and
R¹⁴ is selected from the group consisting of H, alkyl, aryl, and alkoxyalkyl.

49. A method of treating metal poisoning, comprising:
a) providing:
i) a subject with metal poisoning, and
ii) a composition comprising a crowned dithiocarbamate; and
b) administering the composition to the subject;
wherein the crowned dithiocarbamate comprises a crown ether-containing group of formula [(CH₂)ₐ—O]_{b}—(CH₂)_{c}, wherein a is at least 2, b is at least 3, and c is at least 2.
